(19) United States
(12) Patent Application Publication
(10) Pub. No.: US 2011/0159000 A1
(43) Pub. Date: Jun. 30, 2011

(54) ANTIBODIES WITH MANNOSE BINDING LECTIN EFFECTOR FUNCTION FOR INHIBITING PATHOLOGIC INFLAMMATORY CONDITIONS

(75) Inventor: Gregg J. Silverman, Encinitas, CA (US)

(73) Assignee: The Regents of the University of California, Oakland, CA (US)

(21) Appl. No.: 13/062,497

(22) PCT Filed: Sep. 4, 2009
(86) PCT No.: PCT/US09/56125

§ 371 Date: Mar. 4, 2011

Related U.S. Application Data
(60) Provisional application No. 61/191,067, filed on Sep. 5, 2008.

(51) Int. Cl. A61K 39/395; C07K 16/18; A61P 9/10; A61P 31/00; A61P 37/00; A61P 41/00; A61P 35/00

(52) U.S. Cl. 424/142.1; 530/388.15

(57) ABSTRACT

The invention provides compositions comprising monoclonal antibodies which have variable regions that bind an antigen exposed on dead or dying cells and have constant region sequence that contains at least one site which is glycosylated. The antibodies have sufficient type and number of glycans that are ligand for mannose binding lectin (MBL), Administration of the antibodies to individuals suffering from a pathological inflammatory condition treats or inhibits the inflammation via recruitment of MBL.

The invention was made with government support under Grant Nos. AI40305 AR50659, and AI068063 awarded by the National Institutes of Health. The government has certain rights in this invention.

Throughout this application various publications are referenced. The disclosures of these publications in their entireties are hereby incorporated by reference into this application.

### BACKGROUND OF THE INVENTION

To defend against infectious agents, yet also guard against autoimmune disease, complex activating and inhibitory pathways have evolved that interconnect the innate and adaptive immune systems. The innate immune system senses for threats by recognizing microbe-associated molecular motifs using limited sets of cellular receptors, such as Toll-like receptors (TLRs), as well as soluble opsonizing factors, such as complement, collectins and C-reactive protein. Some of these receptors also bind to stress-associated ligands and self-antigens, of which some have been implicated in autoimmune pathogenesis (reviewed in¹). Professional phagocytic cells, macrophages (Mφ) and dendritic cells (DC), thereby, respond to environmental stimuli, self-antigens and cytokines that facilitate or forbid differentiation changes, which determine their capacity to evoke overall inflammatory responses as well as antigen immunogenicity for the adaptive immune system. Overexuberant and excessive responses from Mφ and DC are believed to be important for both the initiation and perpetuation of inflammatory diseases, such as atherosclerosis, as well as autoimmune diseases.

While the innate immune system is important or even essential for modulating lymphocyte responses, innate immune responses themselves are also reciprocally influenced by specialized tiers of the adaptive immune system, such as natural killer (NK), NKT, and γδ T cells, which can recruit DCs into pro-inflammatory responses². There are also cells within the B-lymphocyte compartment that secrete certain antibody products that may have regulatory roles through effects on innate immune cells at remote sites in the body, although how this might occur has not been previously known.

The prototypic T15 B cell clonotype, defined by H-L paired canonical antibody gene rearrangements without hypermutation, was first characterized 40 years ago³. T15 clonotypic B cells spontaneously arise and become highly represented within the first week of life with a similar frequency in mice raised under germ-free conditions⁴, which suggests that microbial ligands are not primary mediators of clonal selection. It is long known that T15-Abs bind to phosphorylcholine (PC) determinants, and contribute to host defense to PC-containing pneumococci, and other microbes, by providing optimal protection from systemic infection⁵. More recently, PC-determinants were reported to be present on oxidatively-modified low density lipoprotein (LDL) generated during atherogenesis⁶. Pneumococcal immunization was reported to induce active B-lymphocyte systemic cellular responses that also raised T15 idiotype antibody (T15-Ab) levels and to ameliorate the chronic inflammatory response in a murine model of hyperlipidemia and atherosclerosis⁷.

Other studies have reported that immune recognition by T15-Ab of the PC head group enables the discrimination of dead/dying cells from healthy cells⁸⁻¹⁰. In healthy cells, the PC-head group, although a ubiquitous component of cell membrane neutral phospholipids (e.g. phosphatidylcholine), is inaccessible for antibody interactions because of being embedded within the lipid bilayer. Apoptotic death-associated membrane alterations, however, expose this PC-head group as neo-determinants that are now recognized by T15-Ab⁸⁻¹⁰. Other antigens are also revealed on apoptotic cells.

Suppression of in vitro and in vivo pathogenic inflammatory response by antibody directed to a PC-determinant (e.g., T15-Ab), phosphatidyl serine (PS) determinant, malondialdehyde (MDA) determinant or cardiolipin determinant, has been described, See WO 2006/086288 published Aug. 17, 2006. As described therein, these antibodies modulate innate immune system responses by suppressing the activation of phagocytes by an inhibitory pathway which is mediated by the antibodies and certain opsonins.

### SUMMARY OF THE INVENTION

Provided herein are compositions comprising purified monoclonal antibodies (including monomeric and/or polymeric antibodies) useful for treatment of pathologic inflammatory conditions. The antibodies of the composition have a variable region that binds an antigen exposed on dead or dying cells. The antibodies of the composition also have a constant region that includes sequence from a heavy chain constant region (preferably human) with at least one site where glycosylation occurs. Thus, the sequence from the heavy chain constant region includes within the sequence at least one site which is glycosylated.

The antibodies of the compositions of the invention are also characterized with respect to their interactions with mannose binding lectin (MBL), a complex protein that recognizes carbohydrate and functions to activate the innate immune system by promoting phagocytosis as well as other functions. The evidence described herein demonstrates the key role played by MBL in reducing pathologic inflammation following administration of antibodies specific for antigenic determinants exposed on dead or dying cells and having sufficient amount and type of glycans which are ligands for MBL and recruit MBL to sites where such antibody targets collections of dead or dying cells in vivo.

Antibodies of the invention are characterized with respect to MBL in two ways. In one way, at least 25% of the antibody molecules that make up a monoclonal antibody composition bind to MBL. Binding to MBL can be determined by any of various methods well known in the art. For example, the antibody composition can be applied to a chromatography column containing immobilized MBL and, following appropriate washing, the percentage of the antibody molecules which are retained by the column (or which are not retained by the column) determined. These fractions are then assayed, for example, for composition based on protein content (e.g. spectrophotometric assay at 280 angstrom) or by immune assay for immunoglobulin content, or by assay for binding capacity for MBL, or by analysis of carbohydrate composition. The percentage of antibody molecules in each individual monoclonal antibody composition that bind MBL are preferably higher than 25%, and may in particular embodiments represent least 30, 35, 40, 45, 50, 55, 60, 65, 70, 75, 80, 85, 90, 95, 98, 99% or even 100%.

Another way that antibodies of the invention are characterized with respect to MBL is by the type and number of glycans which are attached to the antibody constant region. If the antibody is a monomeric antibody, then the monomeric antibody will have, on average, at least one glycan that has dual ligands that bind MBL. When the antibody is a polymeric antibody, then the polymeric antibody will have, on average, at least four glycans that bind MBL. Monomeric antibodies preferably have 2 or more glycans that have dual ligands that bind to MBL while polymeric antibodies preferably have at least 6 glycans that bind MBL. Glycans that have dual ligands is a reference to a branched glycan wherein each branch can contain an accessible ligand for MBL.

The antibodies described herein may have a constant region that includes at least one constant region domain or constant region tailpiece from a human immunoglobulin. Preferably, the constant region domain includes at least one of a CH1, CH2, CH3, or CH4 domain. These domains may be from any Ig isotype and preferably are from an IgM, IgG or IgA.

The constant region of the invention antibodies may have native sequence or may have a variant sequence. The constant region of the antibodies may have less than a full length constant region or may have a full length constant region.

Antibodies of the invention may be fragments of antibodies provided that the variable region has the requisite antigen binding specificity and the constant region that contains one or more sufficient sites that are glycosylated and that allow for recognition by MBL. The antibody may be a full length antibody or may be a fragment of an antibody such as a Fab molecule or F(ab′)₂ molecule. Another antibody fragment can have as the variable region an Fv or single chain Fv configuration.

Antibodies of the invention preferably have constant region sequence that includes not only sufficient sites for attaching glycans with MBL binding ability but also have sufficient sequence for mediating binding by C1q. In a preferred embodiment, the antibodies have a constant region that, whether or not the variable region complexes with its target antigen, the constant region of the antibody lacks affinity for one or more Fc receptors. Alternatively, the constant region may have a constant region sequence that is modified or mutated such that it exhibits reduced affinity for one or more Fc receptors relative to the unmodified or unmutated constant region sequence. A modified constant region sequence that has reduced affinity may involve modification in glycoslation. Sequences in the constant region that play role in C1q binding and/or recognition by Fc receptors are well known as well as variations in natural sequence that enhance or reduce such binding activities. For example, Raju¹¹, describes the impact of Fc glycan terminal sugars on C1q binding. Sensel et al.¹² describes differences in human IgG subtypes for C1q binding and creation of mutants in the CH2 domains of IgG2 and IgG3 with respect to complement receptor interactions including complement dependent lysis of targets. Xu et al.¹³ describes mutations in CH2 residue Pro331 as contributing to the architecture of the IgG1 C1q binding site and that its replacement by a serine residue in IgG4 largely inactivates C1q function in IgG4. Arya et al.¹⁴ describes mutations in the CH3 domain of IgM which impact complement mediated cytolysis. Idusogie et al.¹⁵ describes K326 and E333, mutations which lie at the extreme ends of the C1q binding epicenter in the CH2 domain of a human IgG and increase C1Q binding and cytolytic activity.

As used herein, “dead or dying cells,” includes cell death or dying by any pathway, included programmed cell death (e.g., through apoptosis), autophagy or non-programmed cell death (e.g., by necrosis or injury).

As used herein, an antigen that is exposed on the dead or dying cells is an antigen that is newly exposed, i.e., exposed only when the cell is dead or dying. The newly exposed antigen may be synthesized and expressed solely in the dead or dying cell or may be an antigen that cryptic or sequestered in a healthy cell but which becomes exposed during the process of dying or at death. Antibodies of the invention have variable regions (e.g., a T15 antibody) do not bind, or exhibit reduced binding to healthy cells.

The antigen on dead or dying cell may comprise any of a phosphoryl choline (PC) determinant^{6;10}, phosphatidyl serine (PS) determinant¹⁶, malondialdehyde (MDA) determinant¹⁷, and cardiolipin determinant^{18;19}. Other functionally equivalent antigens or determinants, that are expressed on dead and dying cells, may be targeted by an antibody or recombinant protein, generated according to this invention, even when the identity of the antigen or determinant is not known. In accordance with the practice of the invention, the compositions of the invention may comprise antibodies that bind one or more of these determinants. For example, some antibodies of the composition may bind one determinant and the other remaining antibodies may bind another different determinant. In another embodiment, the composition comprises antibodies that collectively bind PC, PS, MDA and the cardiolipin determinants.

In one embodiment, the antibody has a variable region that recognizes and binds phosphorylcholine (PC) determinants exposed on the dead or dying cell. For example, the antibody that recognizes and binds the PC determinant may be a T15 antibody or a functionally equivalent antibody. Additional examples of antibodies that bind PC include but are not limited to E03, E04, EO6, EO7, EO12, and EO14) as described by Shaw et al.⁶

As used herein, “T15 antibody or variant or fragment thereof” means the T15 antibody, or any antibody or variant or fragment thereof that comprise the same or closely related variable regions of the T15 antibody as described by Shaw et al.⁶. A variant is a molecule that shares sequence similarity and activity of its parent molecule. For example, a variant of T15 antibody includes a molecule having an amino acid sequence at least 80% similar to the variable domain of T15 antibody, encoded by the S107.1 heavy chain variable region gene and which recognizes and binds PC and/or other phospholipid derived determinant. A variant means any change to the amino acid sequence and/or chemical quality, of the amino acid e.g., amino acid sequences that are different from that encoded by the T15 sequence. The antibody can be polyclonal, monoclonal, chimeric, or humanized.

Antibody compositions of the invention are envisioned for treating an individual with a pathologic inflammatory condition are formulated for human use. Such a pharmaceutical formulation may include, in addition to the antibody, other ingredients, for example, water and/or other solvents, salts, buffers, surfactants, and stabilizers.

The antibody compositions of the invention preferably include a single population of monoclonal antibodies but may also include a blend of 2 or more and preferably from 2-4 additional monoclonal antibodies, each of the additional antibodies having the characteristics of the antibodies of the invention. When a blend of multiple monoclonal antibodies is used, the antibodies may have variable regions with specificity for the same antigens or may bind to different antigens exposed on dead or dying cells. For example, all the antibodies in the blend may have variable regions that bind to the PC determinant while another blend may include one antibody to the PC antigen, one to the PS antigen, another to the MDA antigen and another to the cardiolipin antigen.

As used herein “inflammation” is a localized reaction of tissue to irritation, injury, or infection, characterized by pain, redness, swelling, and sometimes loss of function, and that serves as a mechanism that can initiate the elimination of noxious agents and of damaged tissue. As used herein, a “pathologic inflammatory condition” means the presence of tissue destruction and release of certain cytokines and chemokines, and abnormal tissue accumulation of cells of the innate and adaptive immune systems leading to inflammation which becomes pathological. This may involve the recruitment of cells to sites of injury or immunization or immune response in a subject, with release of inflammatory mediators that can include, but are not limited to, certain types of cytokines, histamine, chemokines, prostaglandins and others.

A pathologic inflammatory condition includes, for example, immune system diseases such as lupus erythematosus, autoimmune nephritis or an autoimmune disease such as psoriasis, lymphocytic angiitis, Hashimoto's thyroiditis, primary myxedema, Graves' disease, pernicious anemia, autoimmune atrophic gastritis, Addison's disease, diabetes mellitus, Goodpasture's Syndrome, myaesthenia gravis, pemphigus, Crohn's disease, sympathetic ophthalmia, autoimmune uveitis, multiple sclerosis, autoimmune hemolytic anemia, idiopathic thrombocytopenia, primary biliary cirrhosis, chronic active hepatitis, asthma, chronic obstructive pulmonary disease, pulmonary hypertension, vasculitis, ulcerative colitis, Sjogren's syndrome, inflammatory arthritis, polymyositis, and scleroderma. Arthritis can include psoriatic arthritis or rheumatoid arthritis or Reiter's syndrome or reactive arthritis or juvenile arthritis.

Additionally, a pathologic inflammatory disease can include atherosclerotic vascular disease, or macular degeneration.

Pathologic inflammatory disease may also include condition associated with organ or bone marrow transplantation (e.g., cells, tissues, or organs). In one embodiment, the transplantation associated disease is graft versus host disease (GVHD). In another example, the transplantation disease is transplant rejection such as renal, cardiac, lung or liver allograft transplant rejection. Pathologic inflammatory disease also may be present in patients with cancer.

As used herein, “inhibition” or “treatment” of a pathologic inflammatory condition means to provide an intervention that ameliorates the symptoms of the condition, reduces the severity of the condition, alters the course of disease progression, and/or ameliorates or cures the basic disease problem. For example, treatment of an autoimmune disease may be accomplished by regulating, modulating or suppressing an immune response (e.g., production of antibodies). Although not wishing to be bound by theory, administration of the invention antibody compositions may inhibit or treat pathologic inflammatory conditions by interfering with the activation of the receptor, signaling pathway or molecule essential for the inflammatory condition, as detected by an art-recognized test (for example, the joint swelling and leukocyte infiltration into the joints as is induced in the collagen induced arthritis model). Inhibition or treatment may be partial or total. In another example, inhibition or treatment of a pathologic inflammatory condition can be detected by determining reduction of inflammatory factors and mediators, such as IFN type I and IFN type II, and related cytokines like IL-12. Inhibition or treatment may be partial or total.

Administration of the invention antibody compositions may also increase levels of anti-inflammatory proteins such as MKP-1 or inhibitors of NF-κB, such as IκBalpha or others.

As used herein, ameliorating or reducing the signs and symptoms of a pathologic inflammatory condition means to reduce the signs and symptoms, thereby improving the condition of the treated individual. Signs and symptoms may include: joint swelling and leukocyte infiltration into joints (arthritis), reduction of factors and mediators associated with auto immunity or inflammatory response, such as IFN type I and IFN type II, and cytokines including IL-12, and interferon-gamma, although the specific cytokines that are overexpressed differs between diseases. In addition, although not wishing to be bound by any theory, the methods and compositions of the invention may inhibit or reduce the signs and/or symptoms of autoimmunity or inflammatory disease in a subject by altering the responses of leukocytes capable of interacting with dead or dying cells from the subject. The subjects may include but are not limited to humans, monkeys, pigs, horses, cows, dogs and cats.

In all method embodiments where antibody compositions of the invention are administered, the individual also may be treated with other agents that treat or inhibit, or ameliorate pathologic inflammatory conditions. Such additional agents are described in U.S. Patent Application Publication 20080160020 by Silverman.

In accordance with the invention methods, the antibody composition administered is in an amount suitable to achieve treatment or inhibition of the condition or alleviate symptoms associated with the pathologic condition. Thus, an “effective amount” of a composition of the invention is an amount that inhibits or reduces inflammation (e.g., reduces the symptoms of inflammation) and/or tissue damage and destruction. For example, an effective amount of a T15 antibody, may be defined as the amount of the binding protein (e.g., antibody) that, when bound to dead or dying cells, promotes removal of the dead or dying cells, from the injured area or elsewhere in the body. These binding proteins may also or instead induce macrophages or dendritic cells or other leukocytes to reduce the expression of pro-inflammatory surface molecules or their release of cytokines or other soluble factors. An effective amount of a composition of the invention can be in a range of about 0.1 mg/week to 40 mg/week; 0.1 mg/week to 5 mg/week; 5 mg/week to 10 mg/week; 10 mg/week to 30 mg/week; 30 mg/week to 35 mg/week; 0.1 mg/week to 100 mg/week; or 0.1 mg/week to 50 mg/week; or 50 mg to 200 mg/week. In another embodiment, a composition of the invention can be administered in an amount of about 50 mg/week or 25 mg twice weekly.

The most effective mode of administration and dosage regimen for the compositions of the invention depends upon the location, extent, or type of the disease being treated, the severity and course of the medical disorder, the subject's health and response to treatment and the judgment of the treating physician. Accordingly, the dosages of the compositions of the invention should be titrated to the individual subject and/or by the specific medical condition or disease.

By way of example, the interrelationship of dosages for animals of various sizes and species and humans based on mg/m² of surface area is well known. Adjustments in the dosage regimen may be made to optimize suppression or modulation of the immune response responsible for disease or after transplantation for graft rejection, e.g., doses may be divided and administered on a daily basis or weekly or biweekly or monthly basis or the dose reduced proportionally depending upon the situation (e.g., several divided doses may be administered daily or proportionally reduced depending on the specific therapeutic situation).

As is well known, the dose of the composition of the invention required to achieve an appropriate clinical outcome may be further reduced with schedule optimization.

### BRIEF DESCRIPTION OF THE FIGURES

FIG. 1. In vivo T15-IgM treatment blunts responses to TLR agonists. (A) Groups of adult C57BL/6 mice received saline, IgM isotype control or T15-IgM, then were challenged with agonists for TLR3 (polyl:C), TLR4 (LPS), TLR7 (SM-360320)²⁰, TLR9 (CpG oligo 1018). After 18 hr, mice were sacrificed and splenic Mφ and CD11c^{hi} DC evaluated, as gated (left panels). Representative histograms and mean fluorescence indices (MFI) depicted for: top value from gray shaded area from naïve mouse; saline pretreatment then TLR agonist challenge dark solid line; isotype control followed by TLR agonist challenge from thick gray dashed line; bottom, T15-Ab followed by TLR agonist, black dashed line. Compared to naïve mice, challenge with TLR agonist induces high expression levels of MHC II and CD86. T15-Ab inhibited in vivo activation of Mφ and DC. (B) T15-IgM or apoptotic-cell treatments inhibits polyl:C induced activation of splenic Mφ (F4/80⁺) and myeloid DC(CD11c^{hi}) from C57BL/6 mice. Groups received buffer alone (PBS), necrotic (Necr.) thymocytes, apoptotic (Apop.) thymocytes, isotype control (Iso IgM), T15 or the indicated combination. Results are shown for individual mice at 18 hr after polyl:C or buffer challenge. Horizontal bars depict mean values for each group. There were no significant differences in the representation of splenic F4/80⁺ Mφ and CD11c^{hi} DC between different treatment groups. (C) T15-IgM or apoptotic-cell treatments inhibits polyl:C induction of serum levels of pro-inflammatory cytokines and chemokines in C57BL/6 mice. Serum levels are depicted for treatments followed by polyl:C challenge as determined by Luminex. For indicated groups, P values from two-tailed t test are shown. Results are representative or pooled from three or more independent experiments.

FIG. 2. T15-Ab protects from inflammatory arthritis. (A) DBA/1 mice were immunized with CII and boosted on day 20. T15-Ab at 2 mg/dose, isotype control, apoptotic thymocytes or necrotic cells (2.5×10⁷) in saline, or saline alone administered weekly. T15-Ab and apoptotic-cell treatments significantly reduced arthritis based on clinical arthritis joint scores compared to control treatments (isotype control, saline and necrotic cells) (P<0.001 by Bonferroni test). The isotype control group was not significantly different than saline-treated group. Data are pooled from two independent studies with separate treatment and control groups of four mice (total N=8). Depicted are mean values+/−SEM. (B) Protective T15-Ab reduces inflammatory cellular infiltrates in CIA. Compared to isotype control treatment at left, T15-IgM anti-PC antibody significantly reduced cartilage and bone destruction (arrowhead), and greatly reduced level of cellular infiltrates (arrow)(40Xmag). Below, knees from control-treated mice had progressive pathologic changes of compromised articular cartilage that is shown with safranin O (bright orange), while T15-IgM provided protection from cellular infiltrates, and cartilage and bone destruction, (C) Histologic arthritis scores are depicted for CIA treatment study, with values derived as previously described²¹. (D) To induce autoantibody mediated arthritis, BALB/c mice were injected with a commercial cocktail of anti-CII antibodies, and data represent sequential measurements from two independent studies with separate treatment and control groups of four mice (total N=8). Weekly T15-Ab infusions significantly reduced arthritis based on clinical scores of joint scores, compared to saline or isotype control-treated mice, with P<0.0022 at the peak d14 response, Depicted are mean values +/−SD. (E) In DBA/1 mice immunized with CII and boosted with CII on d20 are representative images of paws of mice after weekly treatment of T15-Ab at 2 mg/dose or control IgM-isotype-treatment groups.

FIG. 3. T15-IgM antibody enhances deposition of C1q and MBL on apoptotic cells and increases their in vivo phagocytic clearance by peritoneal Mφ (A) To assess for C1q deposition, etoposide-treated apoptotic thymocytes were incubated in 50% muMT sera in the presence of saline or purified monoclonal IgM (20 ug/ml) and then washed before staining with 7AAD (to assess membrane integrity) and either labeled anti-murine C1q or isotype control, as indicated. While labeled Annexin V was used to document apoptosis, it was otherwise omitted to avoid interference with C1q-binding. After gating on early (i.e., 7AAD-negative) apoptotic cells, low level C1q binding is shown after muMT sera incubation in the presence of saline or IgM isotype control, while T15-IgM induces a more than 3-fold increase in C1q deposition. At bottom, control studies demonstrated no significant signal (left) on apoptotic cells with the biotinylated isotype control detection reagent, (middle) in the absence of MuMT sera incubation (no sera), or (right) for C1q deposition onto freshly isolated live cells in the presence of T15-Ab. (B) ELISA studies detected interactions with biotinylated recombinant human MBL, with different solid phase precoats when T15-IgM or isotype control IgM were added at 2 ug/ml. Specific MBL- and antigenic-binding of T15-IgM is demonstrated for PC-albumin but not to albumin alone. Specific MBL binding is blocked by mannose or N-acetylglucosamine (NAcGlu) but not N-acetyl galactosamine (NAcGal) at 20 mM. While other assays were in the presence of CaCl₂, the absence of significant binding in 10 mM EDTA-containing buffer confirms the calcium dependence of MBL-binding. (C) Etoposide-treated or γ-irradiated apoptotic thymocytes were incubated with human, recombinant MBL in the absence or the presence of purified monoclonal IgM, then stained with labeled anti-human MBL. At top, binding is shown after gating on early (7AAD-Annexin V+) apoptotic cells. At bottom left, control studies demonstrated no significant signal on apoptotic cells with the biotinylated isotype-control detection reagent. (D) To assess for the capacity to recruit murine MBL from sera, etoposide-treated thymocytes were incubated with MuMT sera in the presence of saline or purified monoclonal IgM, as indicated, Staining was then performed for IgM, and with labeled anti-mouse MBL A and MBL C. In control studies (bottom), background reactivity is seen after incubation of apoptotic cells sera and T15-IgM with staining with an isotype control reagent (left), Incubation without sera yielded background signal with anti-MBL A+C detection, while MBL deposition was greatly reduced by the addition of mannose (50 uM) to sera and T15-IgM. T15-IgM and sera incubation did not result in MBL deposition on healthy cells (right). (E) In vivo Mφ mediated apoptotic clearance was evaluated in three independent experiments, with 2-4 mice in each group that received either T15-IgM, isotype control IgM or saline treatment before instillation of apoptotic thymocytes (total N=9-10/group): MuMT mice (open triangle) that are B-cell deficient; RAG1 ko (open circles and solid circles) that are both B-cell and T-cell deficient, with consistent findings between experiments. Values are presented for each mouse that represent the proportion of peritoneal Mφ recovered after 10 minutes that had engulfed labeled apoptotic thymocytes. (F) T15-Ab enhanced phagocytic engulfment by Mφ of apoptotic cells is shown in cytospin preps from peritoneal cells from B-cell deficient mice that received IgM or saline, 16 hr later were i.p. instilled SNARF-1 fluorochrome (red color) labeled thymocytes, then sacrificed 10 min later, Mφ were detected by F4/80 FITC (green color). IgM treatment is indicated at top, and treatment with either apoptotic or healthy freshly isolated thymocytes, as indicated below. Results are representative of three or more independent experiments. (G) T15-Ab enhances the clearance of both early and late apoptotic thymocytes. Treatment of B-cell deficient mice with T15-IgM but not isotype control enhances in vivo clearance of apoptotic thymocytes. Mice received 1 mg IgM (T15-Ab or isotype control) or saline i.v. 16 hr before i.p. instillation of fresh thymocytes or apoptotic thymocytes. At 10 min after thymocyte instillation, peritoneal cells were recovered with ice cold HBSS/EDTA, and flow cytometric analyses using staining for CD3 (for thymocytes/T cells), and to identify dying cells with 7 AAD and Annexin V (top panel). To evaluate clearance of instilled dying cells, after gating on CD3+ cells that primarily identified transferred thymocytes, the clearance of thymocytes at early stages (7AAD−AnnexinV+) and late stages of death (7AAD+AnnexinV+) were evaluated. Values represent the proportion of mononuclear peritoneal CD3+ cells at early (Annexin V+7AAD−) and late (Annexin V+7AAD+) stages of death from a representative mouse in each group. T15-Ab greatly enhanced clearance of early apoptotic cells compared to saline/negative control treatment (P=0.002), or isotype control (P=0.004). T15-Ab also reduced the representation of dying cells in mice that received only fresh cells (i.e., background level shown at top). In mice that received freshly isolated “healthy” cells, the representation of dying endogenous cells was also reduced by T15-Ab. There were no differences in the numbers of overall recovered peritoneal cells. Four or more mice in each group were evaluated.

FIG. 4. Apoptotic cells induce antibodies to PC-determinants and also to antigens other than PC. Naïve adult C57BL/6 (B6) and congenic S107.1−/− (S107) mice²² were given i.v. infusions of 2.5×10(7) congenic thymocytes undergoing apoptotic death on day 0, 7 and 14. Pretreatment (-pre) and post-treatment (-Apo) bleeds were obtained. Monoclonal anti-PC T15 IgM was used as a standard in certain studies. (A) Representative prebleed and post-immune blood samples were assessed for IgM binding activity to PC-BSA, pneumococcal C-polysaccharide (PS) in which the PC-determinant is immunodominant, copper oxidized low density lipoprotein (OxLDL) or LDL which has been derivatized with MDA (MDA-LDL). Results are indicated for binding activity (OD displayed in the Y-axis, and for serial dilutions indicated in the X-axis. have the same properties. C57BL/6 mice have significantly increased post-immune responses to PC- (PC-BSA and C-PS) antigens and also to Ox-LDL and MDA-LDL. S107.1 VH gene segment deficient mice have no increases in post-immune responses to PC-determinants but nonetheless have induction of IgM levels that recognize OxLDL and MDA-LDL. (B) The same sera at serial dilutions (i.e., 20%, 5% and 1.25%) were incubated for 30 minutes with unfractionated (i.e., total) apoptotic cells, and in some samples incubation was performed along with MDA-BSA-, PC-BSA or BSA at 10 ug/ml, prior to staining for flow cytometric analysis. At top are results for C57BL/6 sera, S107.1 deficient mice at bottom. (C) Comparable studies are depicted, after gating on early apoptotic thymocytic cells (Annexin V+7AAD−). At top are results for 20% C57BL/6 sera, naïve or post-immune. Middle panels are 20% S107.1 deficient mice. At bottom, apoptotic cells were instead incubated with T15 IgM at 20 ug/ml and 20% muMT (Ig-deficient sera as a source of MBL) sera or with muMT sera alone. To demonstrate the specificity of the rat anti-MBL A+C detection antibody, at right bottom used a rat isotype control. Incubation of apoptotic cells with T15 IgM and muMT sera, then developed with an isotype control rat IgG.

FIG. 5. Cultures of purified BM-derived CD11c+ immature DC contain T15-Ab coated apoptotic cells, which are identified by characteristic morphologic changes of nuclear condensation, but not viable cells. The phagocytic capabilities of these cultured DC enables engulfment of cells that spontaneously die in culture, which was confirmed by Annexin V and TUNEL staining. Apoptotic cell-T15-Ab complexes, identified by APC fluorescence, are engulfed by viable DC via pseudopodia lined with CD11c. Under these same conditions, apoptotic cells were not identified by the IgM-isotype control.

FIG. 6. T15-Ab treatment blunts in vitro DC responses to TLR agonists. (A) CD11c+ selected myeloid DC were cultured in replicate with agonists for TLR3 (polyl:C), TLR4 (LPS), TLR7 (imiquimod), or TLR9 (PT CpG oligo 1018), without or with T15-IgM or isotype control at indicated concentrations. Histograms of MHC II and CD40 on DC after culture without or with stimulant (indicated above panel) are depicted and mean fluorescence intensity listed without and with IgM, with concentrations indicated. T15-IgM dose-dependent inhibition of induced expression of these co-stimulatory molecules is shown. (B) From myeloid DC supernatants, levels of pro-inflammatory cytokines and chemokines were determined from standard curves, with values depicted as mean+/−SD, Results are shown without (none) or with stimulants (polyl:C, pIC; imiquimod, imiq) without or with T15-IgM, isotype IgM control, at indicated concentrations. (C) Transcript levels were determined by real time PCR for murine BM-derived CD11c⁺ DC, under indicated cultured conditions over time (min). DC were preincubated with T15-Ab or isotype control before time “0” sampling, then LPS was added. Amplification for TGFβ is β1 isoform-specific. (D) Immunoblots of DC extracts were performed for the indicated conditions at sequential time points (min). Comparisons are for LPS alone, LPS and T15-Ab, or LPS and IgM isotype control. (E) Immunoblots of DC extracts were performed after incubation with LPS alone, LPS co-culture with T15-Ab, or LPS with dexamethasone (Dex). Actin levels confirmed loading levels. Results are representative of three or more independent experiments.

FIG. 7. MKP-1 is induced in DC by co-stimulation with a TLR agonist and T15-Ab. (A) Immunoblots of DC extracts demonstrate that induction of MKP-1 by co-culture of LPS with T15-Ab is blocked by addition of triptolide. With this blockade of MKP-1 protein expression, there were increased levels of LPS-induced p38 phosphorylation, which suggests that T15-Ab inhibits p38 activation through induction of MKP-1, Treatments had limited effects on total MAPK protein. Actin levels confirm loading levels. (B) MKP-1 transcript levels, normalized to a housekeeping gene, were determined after DC stimulation with polyl:C. Controls as indicated, included T15-Ab alone that had only limited early effects on MKP-1 expression.

FIG. 8. T15 IgM enhances phagocytosis by immature dendritic cells of apoptotic cells in an MBL dependent manner. After 5 days in culture with GM-CSF and purification on anti-CD11c-magnetic beads, bone marrow derived immature dendritic cells (iDC) from C57BL/6 mice were put in culture in serum-free media, with CFSE-labeled congenic apoptotic thymocytes. As indicated, replicate cultures included purified T15 IgM or isotype control (NC-17D8) at 20 ug/ml or none, without or with purified recombinant human MBL at the indicated concentration. At top is shown a representative result and gate used to remove unassociated apoptotic cells. At bottom replicate culture results after gating to show only dendritic cells, based on CD11c staining. The boxes show the percentage of DC that have ingested CFSE labeled apoptotic cells after 1 hour. Highest levels of iDC with ingested apoptotic cells were found with T15 IgM and MBL at 20 ug/ml. Notably, iDC themselves produce low levels of MBL²³, which likely contributes to apoptotic cell phagocytosis in the absence of added recombinant MBL.

FIG. 9. Immunoglobulin glycan structures that are ligands for MBL.

FIG. 10. Induction of MKP-1 protein in murine bone-marrow derived dendritic cells (DCs) by T15 IgM and by T15 IgG3, which is dependent on MBL, but not by isotype controls, Bone marrow derived DCs were generated from bone marrow of C57BL/6 in culture by standard methods using GM-CSF and IL-4 for 5 days, Magnetic beads with anti-CD11c were used for isolation to greater than 90% purity. DC were then cultured at 37C with 5% CO2 in serum-free media (Stemspan SF Expansion, (Stem Cell Technologies, Vancouver Canada), without or with LPS at 0.1 ug/ml. Certain replicate wells were preincubated for 60 minutes with purified IgM (20 ug/ml) or IgG (80 ug/ml), without or with purified recombinant MBL at 20 ug/ml and/or C1q (80 ug/ml), prior to the addition of LPS for 30 minutes, then placed at 40. Analysis by flow cytometry with a FACScalibur (Becton Dickinson, Mountainview Calif.) was then immediately performed using intracellular staining with APC conjugated anti-MKP-1 antibody (Becton Dickinson) after gating on MHC II high cells, following manufacturers protocol. Representative data are depicted. A) Shows the gate on MHC II high DCs, and the representation of this subset was the same in all replicate wells and did not change during the period of stimulation. B) Shows the low level expression of MKP-1 in DCs cultured without the LPS stimulant, C) Demonstrates that MKP-1 is increased by LPS, either alone (at left) or with the addition of MBL and C1q together, or MBL or C1q, which caused little or no change in MKP-1 expression. The upregulation of MKP-1 by LPS stimulation alone was expected, and pilot studies confirmed was maximally expressed under these conditions at 30 minutes. D) With LPS stimulation, the addition of purified T15 IgM at 20 ug/ml (left) induced little additional increase of MKP-1, while there was dramatic increases in MKP-1 expression with MBL+C1q together, or with MBL or C1q. E) With LPS stimulation, the addition of an IgM isotype control resulted in little or no increase of MKP-1 expression, even when MBL and/or C1q were also added, F) With LPS stimulation, the addition of recombinant purified T15 IgG3 at 80 ug/ml (left) induced additional increases of MKP-1 expression, while there were further dramatic increases in MKP-1 expression with MBL+C1q together, compared to either MBL or C1q individually. G) With LPS stimulation, the addition of an IgG3 isotype control resulted in little or no increase of MKP-1 expression, even when MBL and C1q were also added. In these studies, the IgM were produced from E06 or IA8 murine B cell hybridomas. The T15 IgG3 was generated by recombinant technology with the identical T15 variable heavy and light chain genes grafted onto a murine gamma 3 and kappa constant regions and expressed in a Chinese Hamster Ovary transfectoma cell line, then purified with Protein A chromatography. Both the T15 IgM and the T15 IgG3 can directly bind MBL, in a calcium depdent and mannose inhibitable fashion, as documented by standard ELISA. Numerical values represent geometric mean fluorescence intensity (MFI) for intracellular MKP-1 staining for gated MHC II high DC, as shown. Higher MFI values present higher levels of intracellular MKP-1 expression.

### DETAILED DESCRIPTION OF THE INVENTION

The invention provides compositions comprising purified monoclonal antibodies (including monomeric and/or polymeric antibodies) or fragments or derivatives thereof useful for treatment of pathologic inflammatory conditions. The antibodies of the composition have a variable region that binds an antigen exposed on dead or dying cells. The antibodies of the composition may bind a single antigen. Alternatively, antibodies of the composition may individually or as a collective bind multiple antigens. The antibodies of the composition also have a constant region that includes sequence from a heavy chain constant region (preferably human) with at least one site where glycosylation occurs. Thus, the sequence from the heavy chain constant region includes within the sequence at least one site which is glycosylated.

The invention also provides methods for treating a disease in a subject resulting from a pathologic inflammatory condition. In this method, the subject is administered a composition comprising a monoclonal antibody that has a variable region that binds an antigen expressed on dead or dying cells and has a constant region that comprises sequence from a human heavy chain constant region that includes at least one site which is glycosylated. In addition, at least 25% of the monoclonal antibodies that make up the monoclonal antibody composition bind to mannose binding lectin (MBL). Alternatively, or in addition, the monoclonal antibody is characterized as to the type and number of glycan moieties that bind MBL. Thus, if the antibody is a monomeric antibody, the monomeric antibody will have on average 2 or more glycans that have dual ligands that bind to MBL while polymeric antibodies preferably have on average at least 4 glycans that bind MBL. Methods to determine whether an antibody has on average a specified number of dual ligands that bind MBL, or have on average a specified number of glycans that bind MBL is readily determined by carbohydrate compositional analysis as described previously^{52,53}.

As used herein, “antibody” includes immunoglobulins which are the product of B cells and variants thereof as well as the T cell receptor (TcR) which is the product of T cells and variants thereof, essentially as described in U.S. Patent Application Publication 20030190676.

As used herein, an immunoglobulin is a protein that comprises a variable and a constant region. A typical immunoglobulin structural unit is known to comprise a tetramer. Each tetramer is composed of two identical pairs of polypeptide chains, each pair having one “light” (about 25 kD) and one “heavy” chain (about 50-70 kD), The N-terminal portion of each chain together defines a variable region of about 100 to 110 or more amino acids primarily responsible for antigen recognition. The terms variable light chain (V_{L}) and variable heavy chain (V_{H}) refer to these light and heavy chains respectively. The variable region of an antibody, as discussed in more detail below, may comprise a full length variable region or a portion of a variable region provided that the portion of the variable region substantially retains its antigen recognition capability.

As used herein, the constant region of an antibody comprises sequence from at least one heavy chain constant region which includes a site where glycosylation occurs. This region may be a CH1, CH2, CH3, or CH4 domain or heavy chain constant region tailpiece. A constant region also may comprise a hinge region. Preferably, the sequence from heavy chain constant region sequence, which includes at least one site which is glycosylated represents a full length constant region domain or full length tailpiece. More preferably, the sequence from a heavy chain constant region which includes a site where glycosylation occurs that represents a full length heavy chain constant region.

Heavy chain constant region sequence may be from alpha, gamma, delta, epsilon or mu constant region. Heavy chain constant region sequence also may be from subclasses of the heavy chain constant region. For example, IgG heavy chains in humans can be any of IgG1, IgG2, IgG3 and IgG4 subclass.

Sequence from at least one heavy chain constant region which includes at least one site which is glycosylated can be identified for any of the constant region immunoglobulin isotypes based on publicly available amino acid and encoding nucleic acid sequence. For example, IgM heavy chain is known to be glycosylated at positions 171, 332, 395, 402 and 563²⁴. The IgG heavy chain known to be glycosylated at position 297 (in CH2)²⁴. The IgD heavy chain is known to be glycosylated at positions 109, 126, 127, 131, 132, 354, 445 and 496²⁴. The IgE heavy chain is known to be glycosylated at positions 168, 218, 140, 265, 394, 371 and 383²⁴. The IgA heavy chain is known to be glycosylated at positions 263 and 459²⁴. Sites for glycosylation may be N-linked (via asparagine) or O-linked (via serine or threonine) type sites.

Monoclonal antibodies of the invention may include a light chain constant region sequence. The light chain constant region sequence may be from kappa or lambda encoded light chains.

Monoclonal antibodies of the invention may be monomeric or polymeric as is well known in the art. Recombinant expression of polymeric antibodies may require coordinate expression of a J chain, Antibody polymers may be an IgA dimer, IgM pentamer or IgM hexamer (the latter lacking a J chain) as well known in the art. Expression of IgA and IgM as polymers is described, for example, in U.S. Pat. No. 5,959,1776; 6,046,037; 6,391,280 and 6,696,620, If co-expression of a J chain is used to obtain a polymeric antibody, the J chain also may one or more sites which are glycosylated.

DNA sequence encoding immunoglobulins variable and constant regions as well as J chains are well known. See for example, Kabat et al.²⁵; and Johnson and Wu²⁶; http://immuno.bme.nwa.edu.

Antibodies exist as full length intact antibodies or as a number of well characterized fragments produced by digestion with various peptidases or chemicals. Thus, for example, pepsin digests an antibody below the disulfide linkages in the hinge region to produce F(ab′)₂, a dimer of Fab which itself is a light chain joined to VH-CH1 by a disulfide bond. The F(ab′)₂ may be reduced under mild conditions to break the disulfide linkage in the hinge region thereby converting the F(ab′)₂ dimer into an Fab′ monomer. The Fab′ monomer is essentially a Fab fragment with part of the hinge region (see²⁷, for a more detailed description of other antibody fragments). While various antibody fragments are defined in terms of the digestion of an intact antibody, one of skill will appreciate that any of a variety of antibody fragments may be synthesized de novo either chemically or by utilizing recombinant DNA methodology. Thus, the term antibody, as used herein also includes antibody fragments either produced by the modification of whole antibodies or synthesized de novo or antibodies and fragments obtained by using recombinant DNA methodologies.

The T cell receptor (TcR) is a disulfide linked heterodimer composed of alpha or beta chains or, on a minority of T cells, gamma or delta chains. The two chains are generally disulfide-bonded just outside the T cell plasma membrane in a short extended stretch of amino acids resembling the antibody hinge region. Each TcR chain is composed of one Antibody-like variable domain (V alpha or V beta) and one constant domain (C alpha or C beta.). The full TcR has a molecular mass of about 95 kDa with the individual chains varying in size from 35 to 47 kDa. Also encompassed within the meaning of TCR are portions of the receptor such as the variable regions of this receptor that can be produced as a soluble protein using methods well known in the art. For example, U.S. Pat. No. 6,080,840 describes a soluble T cell receptor (TcR) prepared by splicing the extracellular domains of a TcR to the glycosyl phosphatidylinositol (GPI) membrane anchor sequences of Thy-1. The molecule is expressed in the absence of CD3 on the cell surface, and can be cleaved from the membrane by treatment with phosphatidylinositol specific phospholipase C (PI-PLC). The soluble TcR also may be prepared by coupling the TcR variable domains to an antibody heavy chain CH2 or CH3 domain, essentially as described in U.S. Pat. No. 5,216,132 or as soluble TcR single chains as described by Schusta et al.²⁸ or Holler et al.²⁹. The TcR “antibodies” as soluble products may be used in place of antibody for making the compounds of the invention. The combining site of the TcR can be identified by reference to CDR regions and other framework residues using the same methods discussed above for antibodies.

Recombinant antibodies may be conventional full length antibodies, antibody fragments known from proteolytic digestion, unique antibody fragments such as Fv or single chain Fv (scFv), domain deleted antibodies, and the like, An Fv antibody is about 50 Kd in size and comprises the variable regions of the light and heavy chain. A single chain Fv (“scFv”) polypeptide is a covalently linked VH::VL heterodimer which may be expressed from a nucleic acid including V.sub.H- and VL-encoding sequences either joined directly or joined by a peptide-encoding linker, See Huston, et al.³⁰. A number of structures for converting the naturally associated, but chemically distinct light and heavy polypeptide chains from an antibody V region into an scFv molecule which will fold into a three dimensional structure substantially similar to the structure of an antigen-binding site, See, e.g. U.S. Pat. Nos. 5,091,513, 5,132,405 and 4,956,778.

The variable region of the antibody comprises a combining site that participates in antigen recognition. The variable region heavy or light chain may be from a human antibody or may be from a non-human antibody (e.g., a murine antibody variable region). A non-human variable region may be “humanized” using methods well known in the art.

The antigen binding site of an antibody is formed by amino acid residues of the N-terminal variable (“V”) regions of the heavy (“H”) and light (“L”) chains. The antibody variable regions comprise three highly divergent stretches referred to as “hypervariable regions” or “complementarity determining regions” (CDRs) which are interposed between more conserved flanking stretches known as “framework regions” (FRs). In an antibody molecule, the three hypervariable regions of a light chain (LCDR1, LCDR2, and LCDR3) and the three hypervariable regions of a heavy chain (HCDR1, HCDR2 and HCDR3) are disposed relative to each other in three dimensional space to form an antigen binding surface or pocket. The antibody combining site for antigen therefore represents the amino acids that make up the CDRs of an antibody and any framework residues that also contribute to the binding site.

The identity of the amino acid residues in a particular antibody that make up the combining site can be determined using methods well known in the art. For example, antibody CDRs may be identified as the hypervariable regions originally defined by Kabat et al.²⁵; http://immuno.bme.nwa.edu. The positions of the CDRs may also be identified as the structural loop structures originally described by Chothia and others,³¹⁻³³. Other methods include the “AbM definition” which is a compromise between Kabat and Chothia and is derived using Oxford Molecular's AbM antibody modeling software (now Accelrys) or the “contact definition” of CDRs by MacCallum et al.³⁴.

The following chart identifies CDRs based upon various known definitions,

| Loop | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 | H31-H35B | H26-H35B | H26-H32 . . . 34 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H52-H56 | H47-H58 |
| H3 | H95-H102 | H95-H102 | H95-H102 | H93-H101 |

General guidelines by which one may identify the CDRs in an antibody from sequence alone are as follows:
LCDR1:
   Start—Approximately residue 24.
   Residue before is always a Cys.
   Residue after is always a Trp. Typically TRP is followed with TYR-GLN, but also may be followed by LEU-GLN, PHE-GLN, or TYR-LEU.
   Length is 10 to 17 residues.
LCDR2:
   Start—16 residues after the end of L1.
   Sequence before is generally ILE-TYR, but also may be VAL-TYR, ILE-LYS, or ILE-PHE.
   Length is generally 7 residues.
LCDR3:
   Start—generally 33 residues after end of L2.
   Residue before is a Cys.
   Sequence after is PHE-GLY-X-GLY.
   Length is 7 to 11 residues,
HCDR1:
   Start—at approximately residue 26 (four residues after a CYS) [Chothia/AbM definition] Kabat definition starts 5 residues later,
   Sequence before is Cys-X-X-X.
   Residues after is a TRP, typically followed by VAL, but also followed by ILE, or ALA.
   Length is 10 to 12 residues under AbM definition while Chothia definition excludes the last 4 residues.
HCDR2:
   Start—15 residues after the end of Kabat/AbM definition of CDR-H1.
   Sequence before typically LEU-GLU-TRP-ILE-GLY, but a number of variations are possible.
   Sequence after is LYS/ARG-LEU/ILE/VAL/PHE/THR/ALA-THR/SER/ILE/ALA
   Length is 16 to 19 residues under Kabat definition (AbM definition ends 7 residues earlier).
HCDR3:
   Start—33 residues after end of CDR-H2 (two residues after a CYS).
   Sequence before is CYS-X-X (typically CYS-ALA-ARG).
   Sequence after is TRP-GLY-X-GLY.
   Length is 3 to 25 residues.

The identity of the amino acid residues in a particular antibody that are outside the CDRs, but nonetheless make up part of the combining site by having a side chain that is part of the lining of the combining site (i.e., it is available to linkage through the combining site), can be determined using methods well known in the art such as molecular modeling and X-ray crystallography. See e.g., Riechmann et al.³⁵. The aldolase antibody mouse mAb 38C2, which has a reactive lysine near to but outside HCDR3, is an example of such an antibody.

Antibodies suitable for use herein may be obtained by conventional immunization, reactive immunization in vivo, or by reactive selection in vitro, such as with phage display. Antibodies may be produced in humans or in other animal species. Antibodies from one species of animal may be modified to reflect another species of animal. For example, human chimeric antibodies are those in which at least one region of the antibody is from a human immunoglobulin. A human chimeric antibody is typically understood to have variable regions from a non-human animal, e.g. a rodent, with the constant regions from a human. In contrast, a humanized antibody uses CDRs from the non-human antibody with most or all of the variable framework regions from and all the constant regions from a human immunoglobulin. Chimeric and humanized antibodies may be prepared by methods well known in the art including CDR grafting approaches (see, e.g., U.S. Pat. Nos. 5,843,708; 6,180,370; 5,693,762; 5,585,089; 5,530,101), chain shuffling strategies (see e.g., U.S. Pat. No. 5,565,332; Rader et al.³⁶), molecular modeling strategies (U.S. Pat. No. 5,639,641), and the like.

Antibodies with binding specificity for antigenic determinants exposed on dead or dying cells can be prepared as described previously in U.S. Patent Application Publication 20080160020 by Silverman. The antigen on dead or dying cell may comprise any of a phosphoryl choline (PC) determinant^{6;10}, phosphatidyl serine (PS) determinant¹⁶, malondialdehyde (MDA) determinant¹⁷, and cardiolipin determinant^{18;19}. In accordance with the practice of the invention, the compositions of the invention may comprise antibodies that singly or collectively bind any one or more of these determinants. For example, the composition of the invention may include antibodies that singly or collectively bind at least two determinants such as PC and PS; PC and MDA; or PC and cardiolipin. Other combinations not including PC are possible. In another embodiment, the composition comprises antibodies that collectively bind all these determinants, Collective binding means one group of antibodies binds one determinant, another group of antibodies binds another different determinant and so on until as a collective, the antibodies of the composition bind some or all of these determinants.

As used herein, the term “purified” in reference to antibodies does not require absolute purity. Instead, it represents an indication that the antibody or antibodies is(are) in a discrete environment in which abundance (on a mass basis) relative to other proteins is greater than in a biological sample from which the antibody or antibodies have come. By “discrete environment” is meant a single medium, such as a single solution, a single gel, a single precipitate, etc. Purified antibodies may be obtained by a number of methods including, for example, laboratory synthesis, chromatography, preparative electrophoresis, centrifugation, precipitation, affinity purification, etc. One or more “purified” polypeptides of interest are preferably at least 10% of the protein content of the discrete environment. One or more “substantially purified” antibodies are at least 50% of the protein content of the discrete environment, more preferably at least 75% of the protein content of the discrete environment, and most preferably at least 95% of the protein content of the discrete environment. Protein content is determined using a modification of the method of Lowry et al., J. Biol. Chem. 193: 265, 1951, described by Hartree, Anal Biochem 48: 422-427 (1972), using bovine serum albumin as a protein standard.

Antibody compositions of the invention are non-natural compositions. That is the antibodies in the composition have been engineered and/or purified from their original source.

As discussed, the antibodies of the invention are characterized in binding to MBL or having a particular type and number of glycans which bind to MBL. MBL is a C-type lectin, which is a protein with the capacity to specifically recognize certain carbohydrates, that include N-acetyl glucosamine (GlcNAc), glucose, fucose, and mannose, but not galactose or N acetylgalactose³⁷. These interactions require calcium and possibly divalent cationic ions for binding. When fully assembled, MBL, which is made up of 18 subunits, is capable of high avidity multivalent interactions, MBL is a secreted molecule present at significant serum concentration (>1 ug/ml in most individuals), and likely at high levels in extracellular milieu as well. It is made in the liver for secretion into plasma, but is also secreted by many local cell types, including macrophages and dendritic cells, MBL can be obtained using recombinant expression as described in U.S. Pat. No. 6,846,649 or may be obtained from natural sources as described in U.S. Patent Application Publication 20050037949.

MBL is the first component of the lectin pathway of complement activation. MBL directly recognizes a variety of pathogenic microorganisms including Gram-positive and -negative bacteria, yeast, mycobacteria, parasites, and viruses.³⁸ By recognition of certain patterns of carbohydrate structures, MBL binding can trigger complement activation to contribute to defense from infection³⁹. MBL released from the liver or from DC or Mφ is reported to be deposited on late apoptotic cells⁴⁰, where it is reported to act as a ligand for LRP-1 on phagocytes⁴¹, or other receptor. MBL-deficient mice have defects in clearance of apoptotic cells, although they do not develop autoimmune disease⁴².

Addition of high levels of MBL to cultured mononuclear cells has also been reported to down regulate inflammatory responses⁴³(reviewed in⁴⁴). MBL is reported to directly bind to apoptotic cells^{41;45}. However, this recognition is predominantly for late stage apoptotic and necrotic cells⁴⁰, These and other apoptotic cell ligands are essential for the stimulated ingestion via macropinocytosis (also termed efferocytosis) by phagocytic cell that evokes an anti-inflammatory response. Under some conditions, there may be associated release of TGF-β or IL-10 (reviewed in⁴⁶), although suppression of NF-κB responses have also been implicated⁴⁷. Addition of MBL can modulate the responses of mononuclear cell responses⁴⁴

The structural basis for MBL binding to immunoglobulins occurs through carbohydrate units attached to the immunoglobulin protein backbone either by N- or O-glycosidic bonds or by both types of linkage. The N-glycosidic bond is to the amide of an asparagine that is part of the consensus sequence NXS/T, where X can be any amino acid except proline. These sites are often referred to as “potential glycosylation sites”, because not all of these asparagine sites are glycosylated.

Carbohydrate is added to the immunoglobulin variable or constant region during synthesis in the cell. While not all sites are glycosylated, and the process may not go to completion, there is a well-characterized sequence of sugar addition by glyco-transferases and sugar removal by glycosidases that leads to a final complete oligosaccharide that terminates with sialic acids (Neu5Ac). However, often the natural process does not fully progress to completion. Reviewed in⁴⁸. Hence the intermediate products may be very different from the final form and even different from simple truncated versions of the final complete complex N-glycan. Overexpression of certain of these enzymes during N-glycan formation or afterwards can therefore change the composition of the final immunoglobulin N-glycan and its capacity to interact with sugar binding receptors such as MBL⁴⁹, There are also specific glycolytic enzymes that can remove terminal residues from fully formed N-glycans, which can also yield products with enhanced MBL-binding properties⁵⁰. A cell line for production of recombinant antibody can also be modified by the use of RNAi technology or be similar means to inhibit the expression of sialotransferase and/or galactosetransferase to also provide a means to produce recombinant protein with N-glycans that have enhanced MBL binding activity.

In practice, N-glycan formation in a B cell or plasma cell or transfectoma can be affected by a wide range of known and unknown factors. For instance changing the media and level of oxygenation can change the relative glycosylation product⁵¹. In addition, the presence of pro-inflammatory factors can result in incomplete glycosylation and relative deficiency in terminal sialic acids and galactoses. All of these factors can alter relative MBL binding activity of an immunoglobulin preparation.

The process of N-glycan formation often generates heterogeneous products. The relative level of completion of the N-glycan product may differ due to differences in the cells used for producing the immunoglobulin. Also, there may be N-glycan differences at multiple glycosylation sites within a single immunoglobulin molecule (e.g., IgM, when formed as apentamer, has ten mu chains which can each be glycosylated to varying extents at each site in each chain, while IgG generally has two gamma chains which can each be variously glycosylated at the single site in most IgG known for glycosylation). An IgG molecule can have additional N-glycosylation sites.

Antibody heavy chains which define the various isotypes are known to differ in their potential to contain MBL binding sites. A subset of IgG, IgM and IgE are known to bind MBL while native IgE binds very poorly and native IgD does not bind. Methods are provided herein for obtaining antibody preparations having a significant fraction containing MBL binding capability.

In some cases, glycosylation sites may be associated with the variable region of an immunoglobulin. The variable region and/or the constant region of an immunoglobulin can be engineered to include asparagine residues that have a glycan which binds MBL. Glycosylation also may be associated with immunoglobulins through polypeptides other than the heavy or light chain. For example, the J chain of IgM also contains a single N-linked glycosylation site at Asn-48. Based on the production conditions, such N-linked glycans may bind MBL.

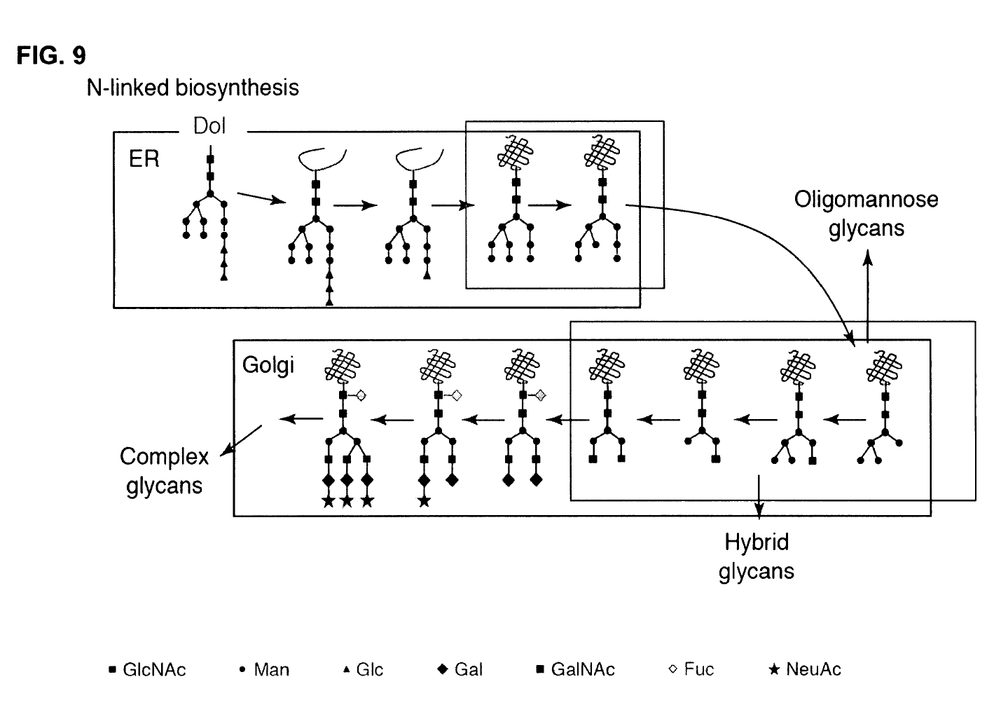

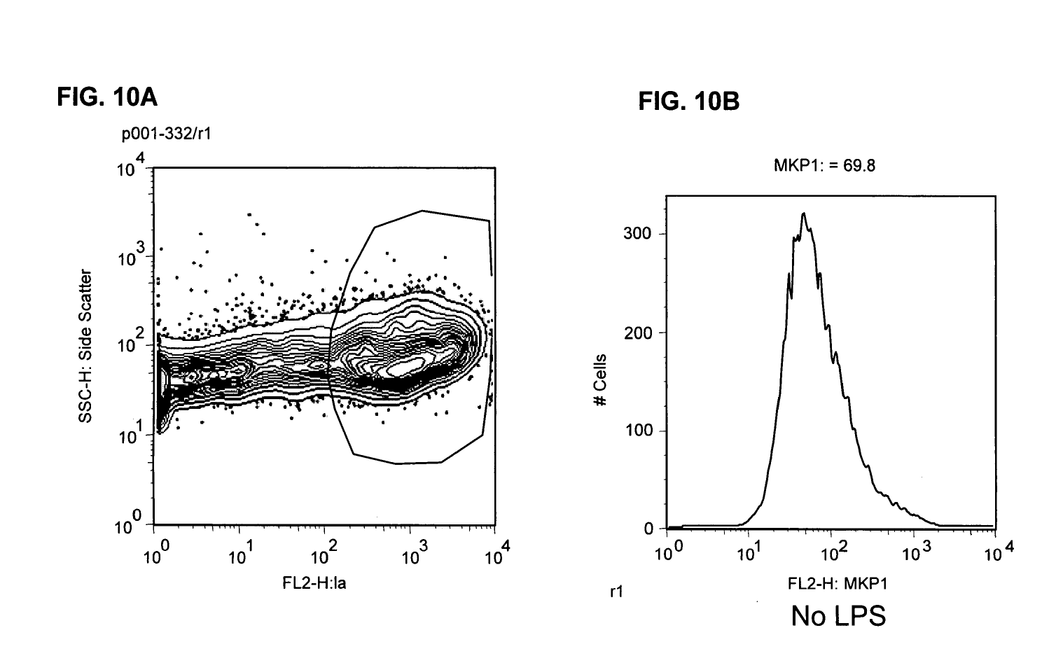

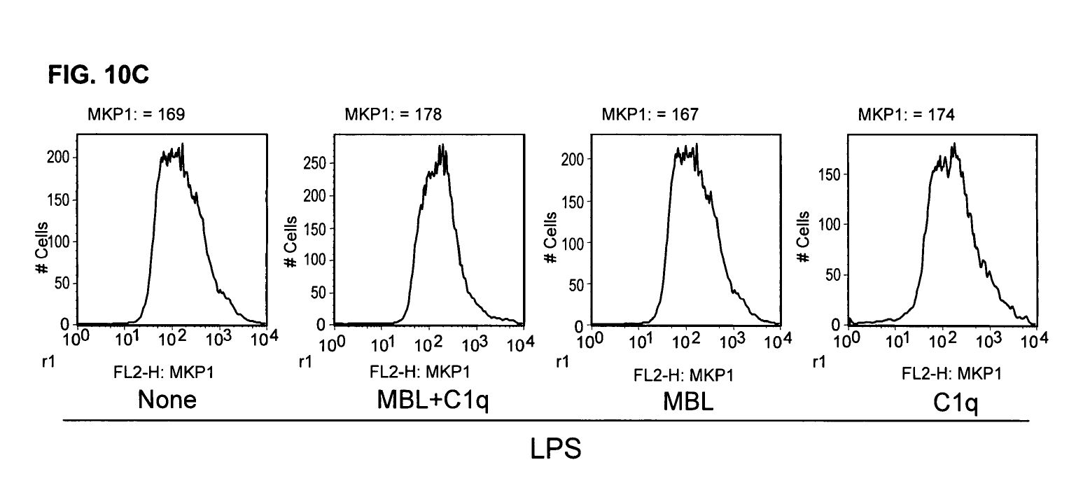

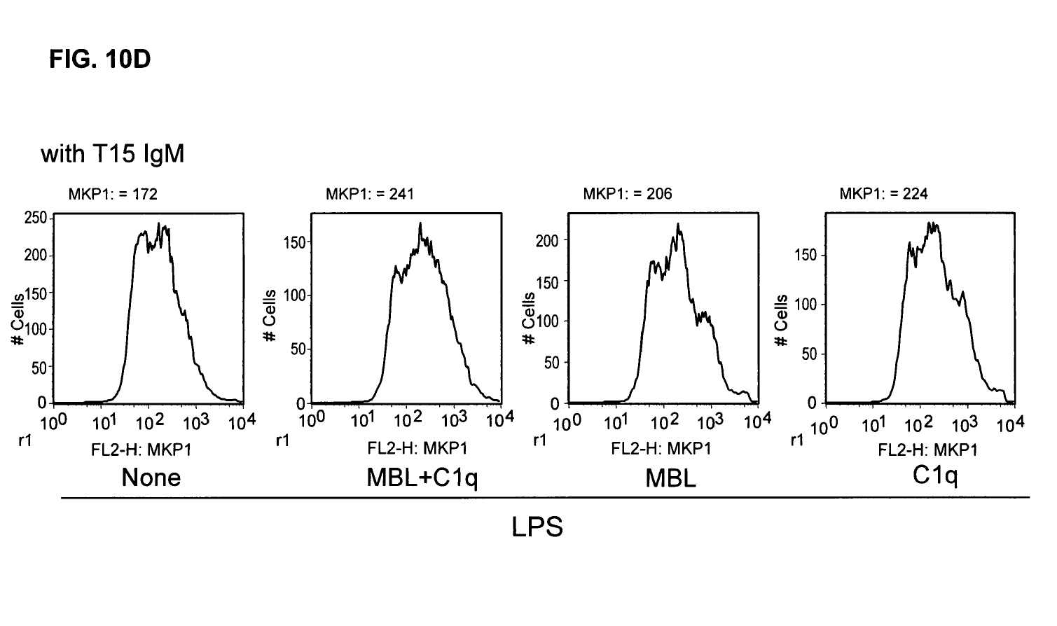

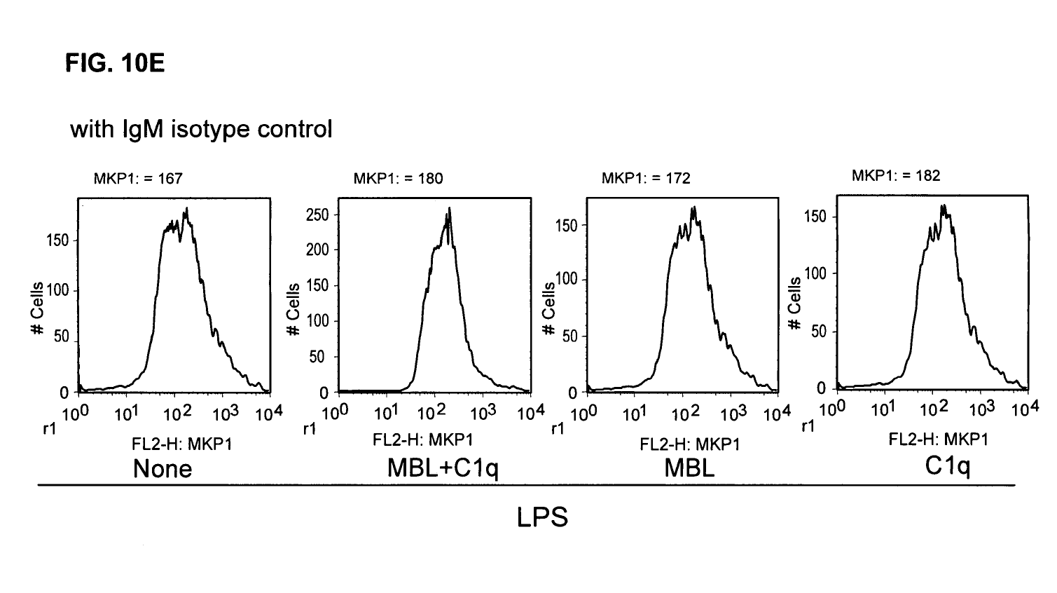

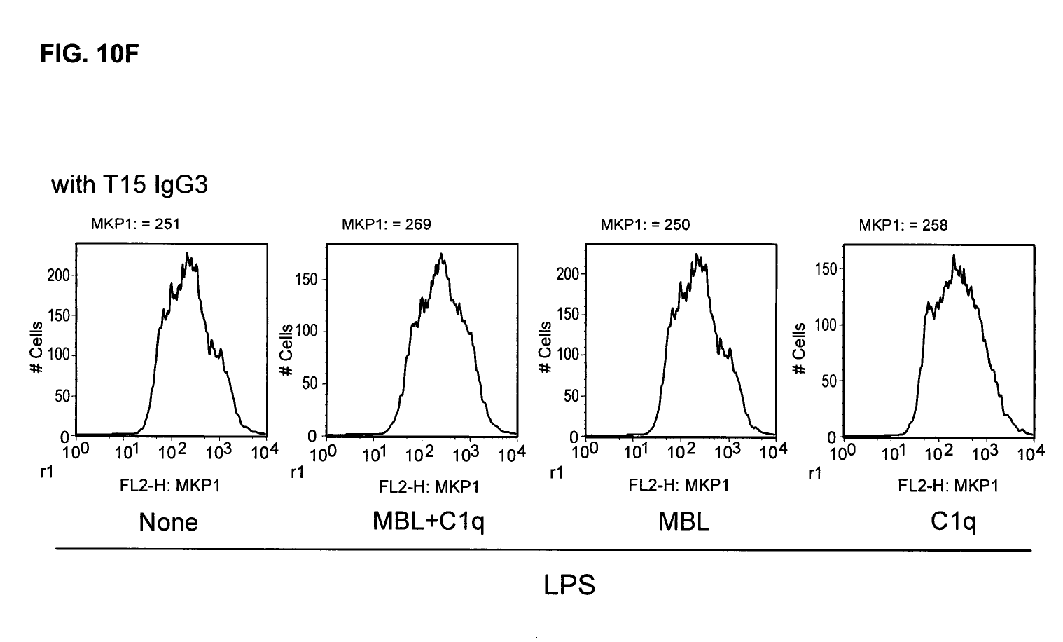

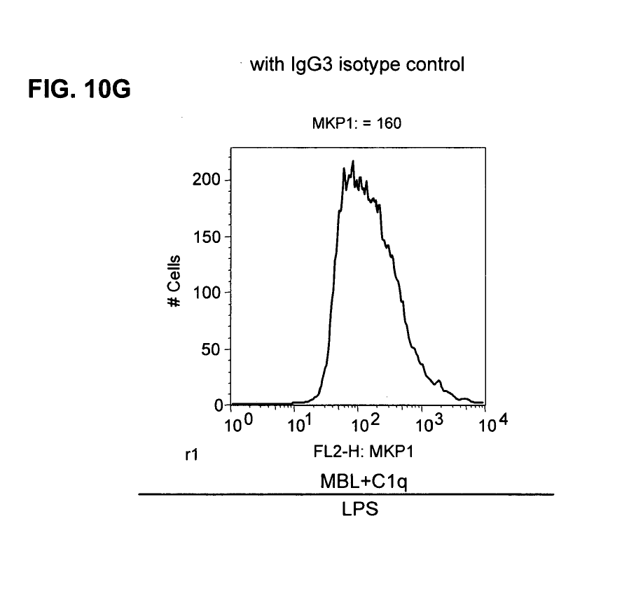

Exemplary glycan structures present on antibody constant regions that are ligands for MBL are shown in FIG. 9. MBL binding glycans in FIG. 9 include those shown as being resident in the ER and those shown in the Golgi as “hybrid glycans.” These glycans are branched and may have one or two MBL ligands per glycan (either one arm branch with an MBL glycan or both branches with an MBL ligand). MBL ligand structures include the two from the right in the ER and the four from the right as depicted here in the Golgi.

IgM Fc has five potential N-linked glycosylation sites on each μ chain, located at Asn-171, Asn-332, Asn-395, Asn-402, and Asn-563, although Asn⁴⁰² present in the hinge region of IgM is unlikely to be accessible for MBL binding. Asn-171, Asn-332, and Asn-395 are occupied by complex glycans, whereas Asn-402 and Asn-563 are occupied by oligomannose glycans.

In general, only a minority (about 20%) of human IgM in circulation is a ligand for MBL⁵². This minority of human IgM that binds MBL is believed to be mediated by GlcNAc terminating glycans present in higher frequency (than other serum IgM devoid of MBL binding activity) at positions 171, 332 and 195 and from an oligomannose glycan with terminal residues of GlcNAc located at Asn⁴⁰²(⁵²).

Each heavy chain of IgG most often has a single covalently N-linked glycan at Asn²⁹⁷ located in the Fc. In the IgG-G0 form, a branched glycan on the CH2 domain lacks terminal sialic acid and penultimate galactose residues, and instead terminates with GlcNAc residues that MBL can bind, Human IgG can greatly vary with regard to MBL binding activity, as inflammatory diseases such as rheumatoid arthritis is known to be commonly associated with higher levels of G0 glycans that bind MBL⁵³. This is believed to result from the high level of inflammatory factors in these patients, which can affect either N-glycan formation on the Ig, or alter the in vivo degradation of these Ig to reveal these MBL binding sites.

The immunobiologic properties of antibodies are known to be affected by N-glycosylation in ways other than MBL recruitment capacity. In general, loss of the terminal sialic acid (Neu5Ac) alone may enhance FcγR-mediated inflammatory responses⁵⁴.

Methods are provided herein for preparing antibody compositions that contain a significant population of antibodies with MBL binding capability and for testing the resulting antibodies for MBL binding.

Method 1. Growth of Antibody Producing Cells Vitro Under Culture Conditions that Enhance MBL Binding Activity with Selective Glycotransferase Inhibitor.

For example, hybridomas or transfectomas producing IgG antibody may be cultured in the presence of glycotransferase inhibitors that will yield IgG with “high mannose” oligosaccharide N-glycans. To alter the final N-glycans of an IgG, the IgG-producing cell line is cultured with swainsonine (available from EMD) to block alpha-mannosidase II to yield the Ig with N-linked core mannose-5 devoid of the capping GlcNAc, Gal or Neu5Ac. Glycoconjugates with potent MBL-binding capacity can also be generated by culturing the hybridoma or transfectoma with deoxy-mannojirimycin (available from EMD), a specific alpha-mannosidase I inhibitor, which blocks the conversion of high mannose to complex oligosaccharides, thus yielding Ig with N-linked mannose-9 glycan.

Method 2. Growing Antibody Producing Cells In Vitro Under Culture Conditions that Favor Incomplete Glycosylation at N-Glycosylation Sites.

Variations in culture conditions for hybridomas and transfectomas that enhance (or diminish) completion of glycosylation has been reported^{51;55}. Culture conditions that do not enhance completion of glycosylation should produce antibodies with greater amounts of terminal sugars that are ligands of MBL.

Method 3. Exposing Antibody to Glycosidases that Remove Terminal Blocking Sugars of the N-Glycan.

The removal of the terminal sialic acid and penultimate galactose from certain glycans with sialidase and beta-galactosidase enzymes results in glycans with greater MBL binding. This can be used for IgM, IgG or for IgA as described previously⁵⁰.

Method 4. Isolation of an Antibody with MBL-High Binding Fraction Using MBL Affinity Chromatography

An MBL enriched fraction of an antibody can be obtained by MBL affinity chromatography as described previously⁵⁶. Commercially available columns with immobilized murine MBL are available (IgM purification kit, Cat No. 44897, Pierce Biotechnology), and have been successfully used to isolate the fraction of an immunoglobulin preparation with the MBL binding activity and determine the representation of this activity⁵², For example, serum or Ig preparation is first dialyzed, or diluted with a calcium-containing buffer and then applied to the column at 4C. The non-binding fraction is removed by washing with the same calcium-containing buffer. Thereafter, the column is washed with a series of buffers that contain increasing amounts of EDTA, a calcium chelating compound. Tris buffered saline has been used as the buffer. In the absence of calcium, the MBL binding proteins are eluted from the column. The protein content, and content of functional antibodies is then measured. In addition, one can measure the amount of antibody applied to the column and the amount which is retained by the column to calculate a percentage of the antibody which binds to MBL.

### Method 5: Determination of MBL Activity of MBL Enriched Antibody Preparations.

(A) The variable region antibody specificity for different PC-antigens, MDA-antigens, or other binding specificity is assessed by standard ELISA. Microtiter wells are coated with the antigen of interest and incubated overnight. Then the contents of the wells are discarded and a blocking solution of irrelevant protein, such as bovine serum albumin, is added to block residual open-binding sites on the wells. The antibody of interest is added in serial concentrations to the wells. After incubation at room temperature for at least four hours, the solutions are discarded and non-binding proteins removed by serial washing in a mild detergent solution (e.g. 0.05% Tween20). A developing reagent specific for the isotype of interest is then added (e.g. horse radish peroxidase (HRP) tagged goat anti-mouse IgM), after 1 hour of incubation, it is removed the wells washed and the developing reagent added. To assess relative activity a standard serial dilution of a positive control antibody is included on the microtiter plate.

(B) The capacity of the antibody to bind MBL can be assessed in vitro by ELISA, using the same general format as above. In this case, all incubations instead use Tris buffered saline (TBS) pH 7.4 with CaCl₂ at 5 mM at all times for incubations and washes and the developing reagent is 1 ug/ml of human recombinant MBL-biotin tagged, with incubation overnight at 4C. Afterward, wells are washed and incubated with HRP-tagged streptavidin. Several variations of this assay have been previously reported⁵⁷.

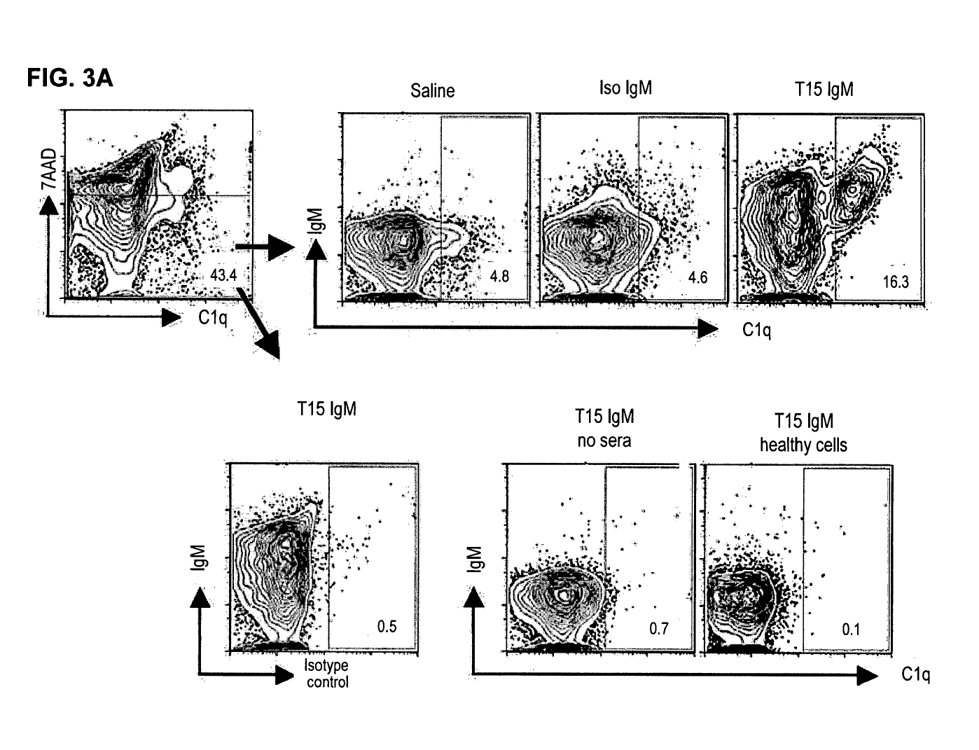

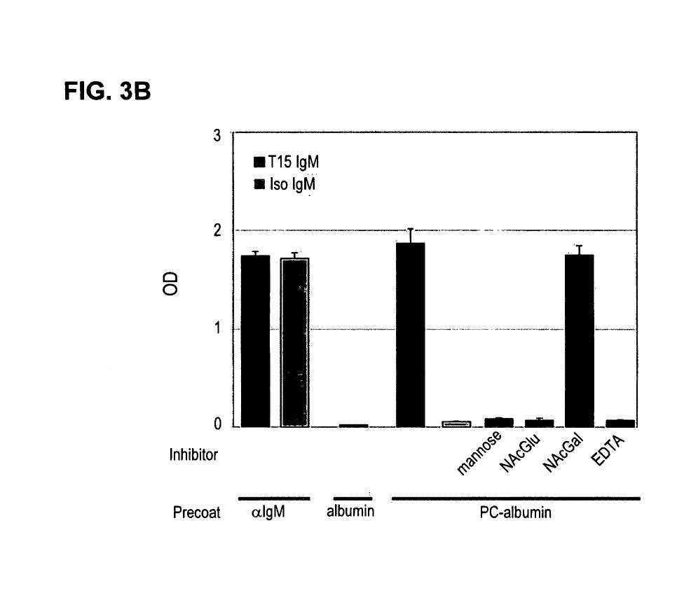

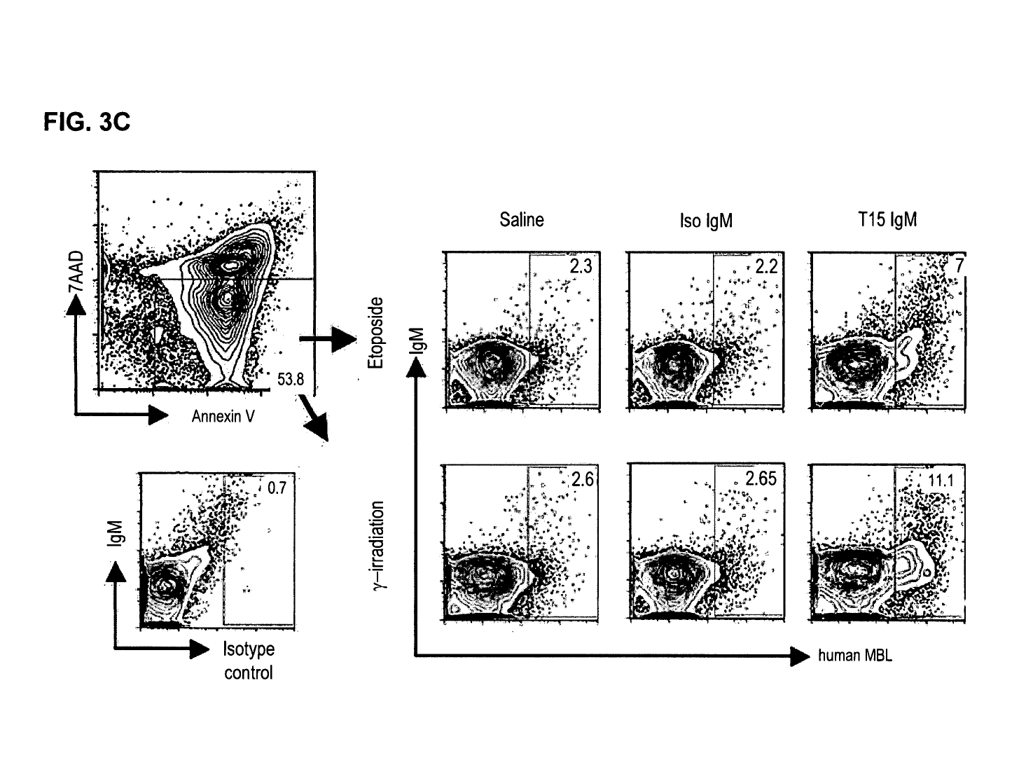

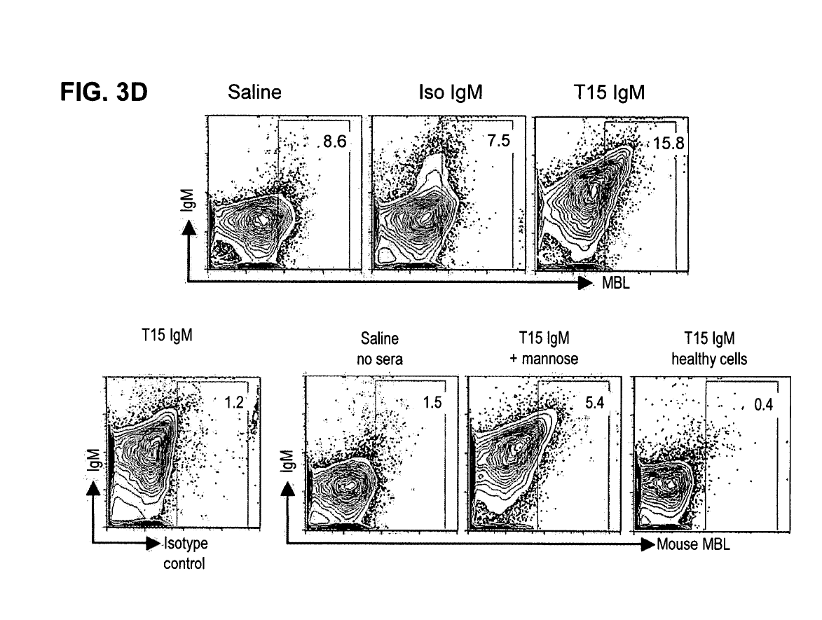

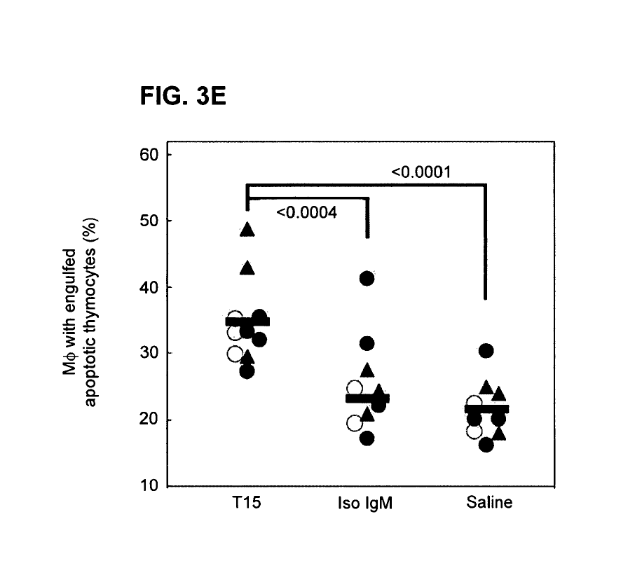

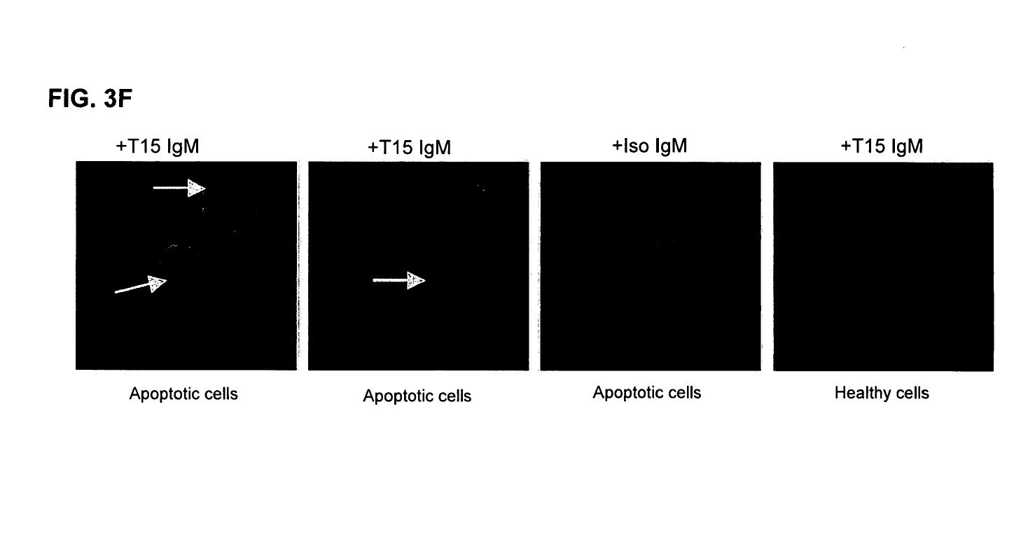

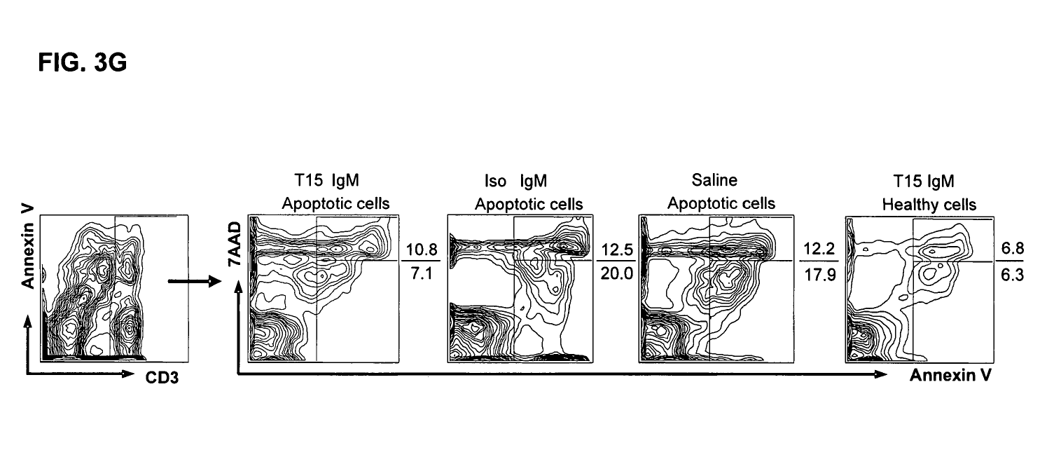

To document mannose-dependence of the interactions, Ig will be treated with jack bean alpha-mannosidase (Sigma), which removes accessible mannose residues and destroys MBL-binding activity. In fact, these treatments can be performed in an ELISA well after the antibody has been bound to precoated anti-Ig or PC-conjugates or other antigen. Specificity of the MBL-interaction may also be demonstrated by showing the requirement for divalent cation, as MBL binding is blocked by 10 uM EDTA in the buffer instead of Ca++. It is also selectively blocked by mannose, GlcNAc at 10-50 mM (culture tested)(Sigma), but is not blocked by GalNAc (see FIG. 3)^{50;58}.

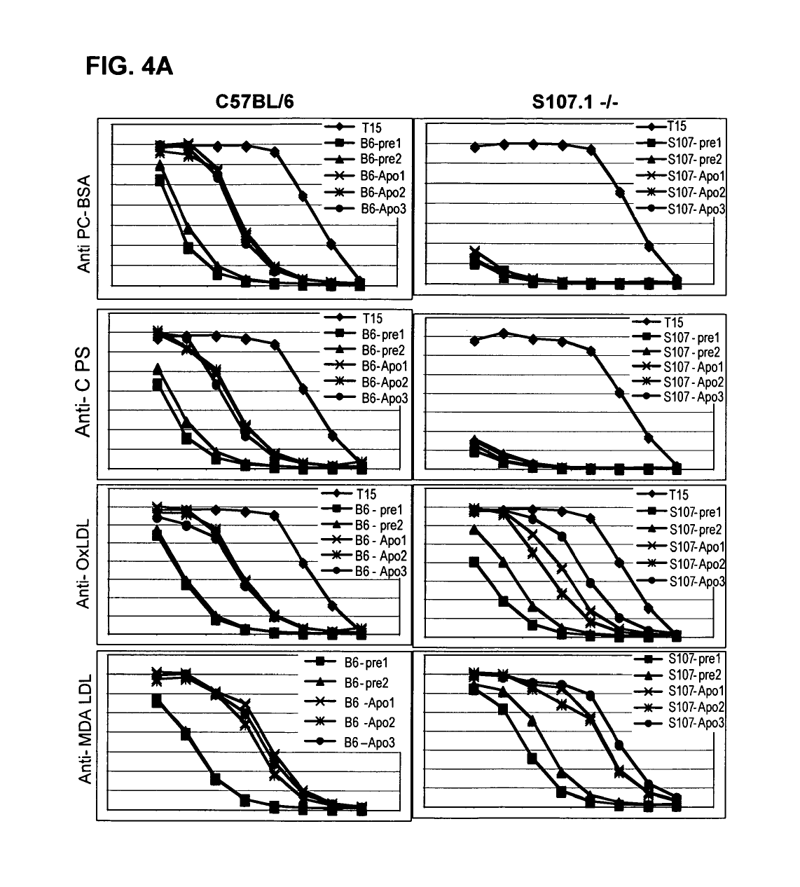

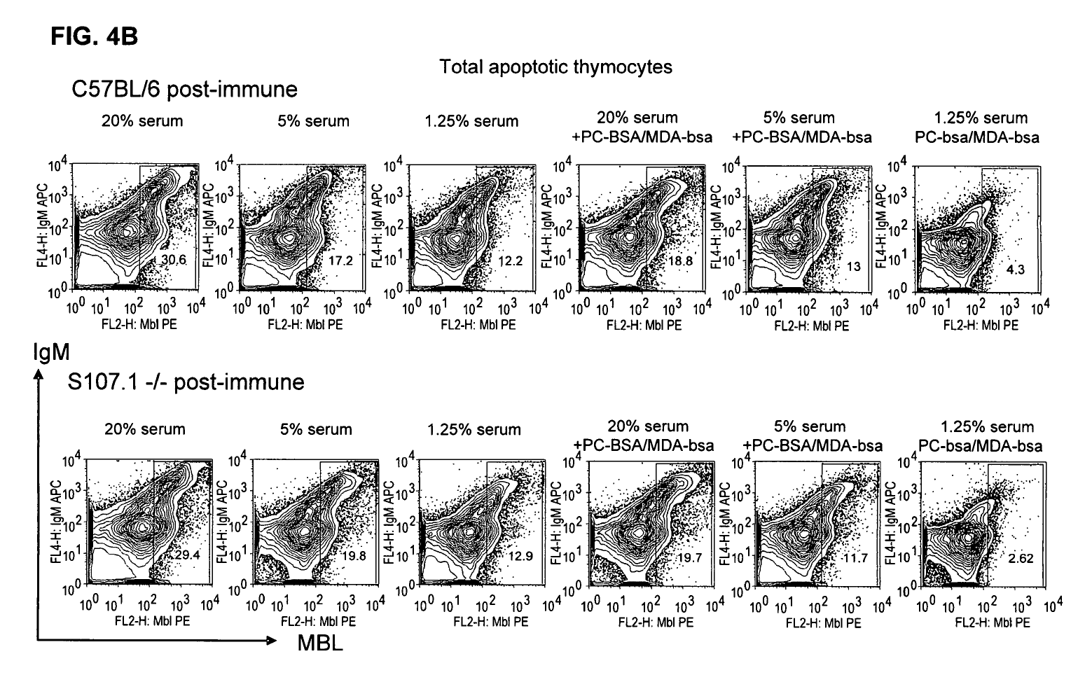

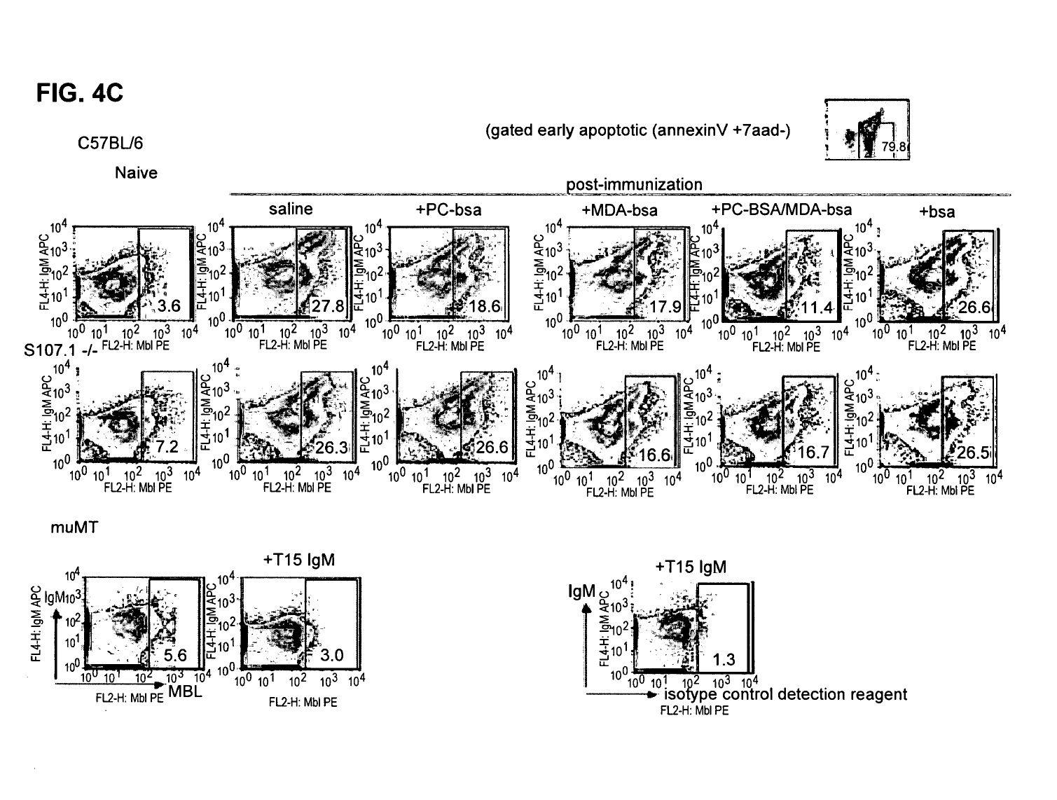

(C) The capacity of antibody to mediate deposition of MBL on apoptotic cells can be demonstrated as described in the Examples and as shown in FIGS. 3 and 4.

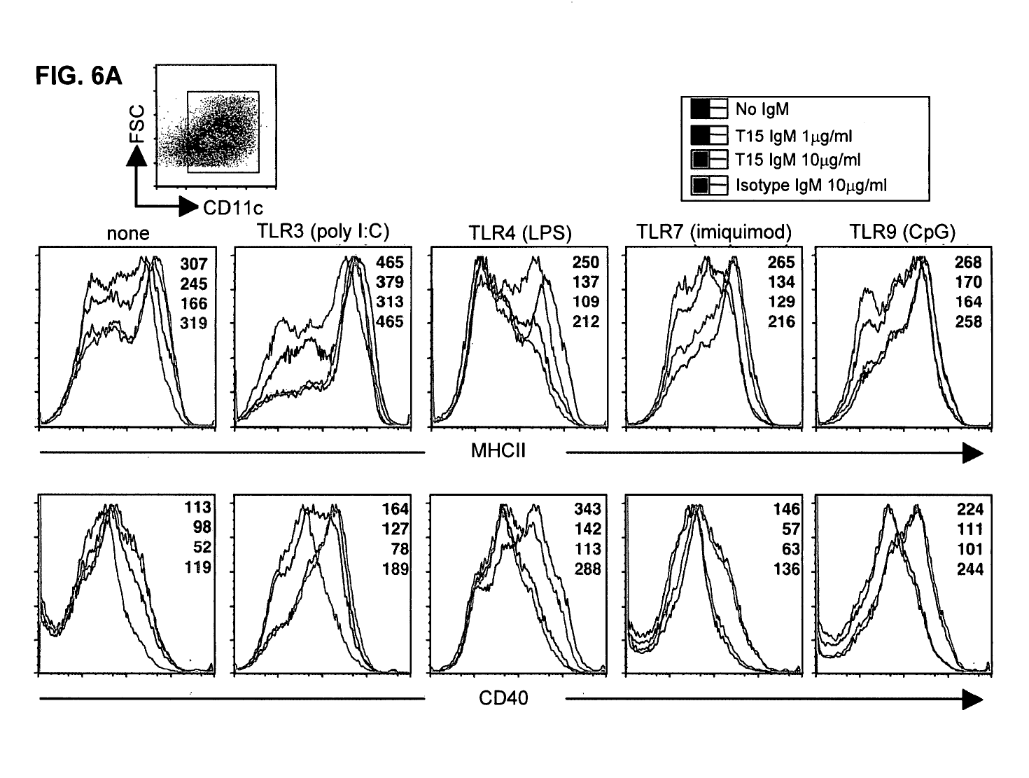

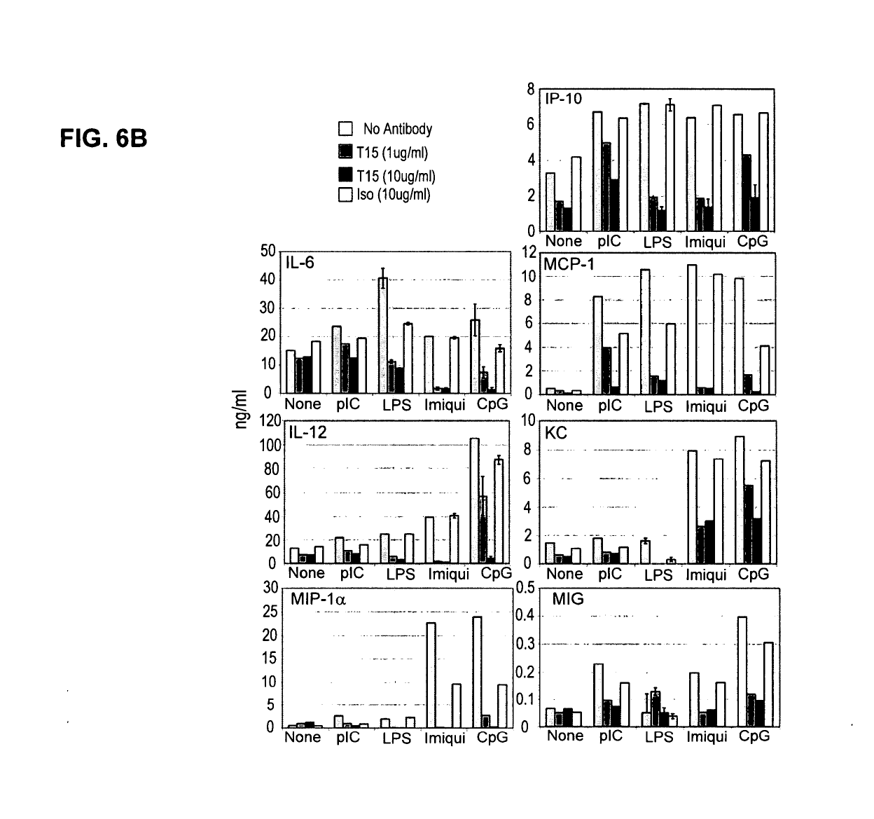

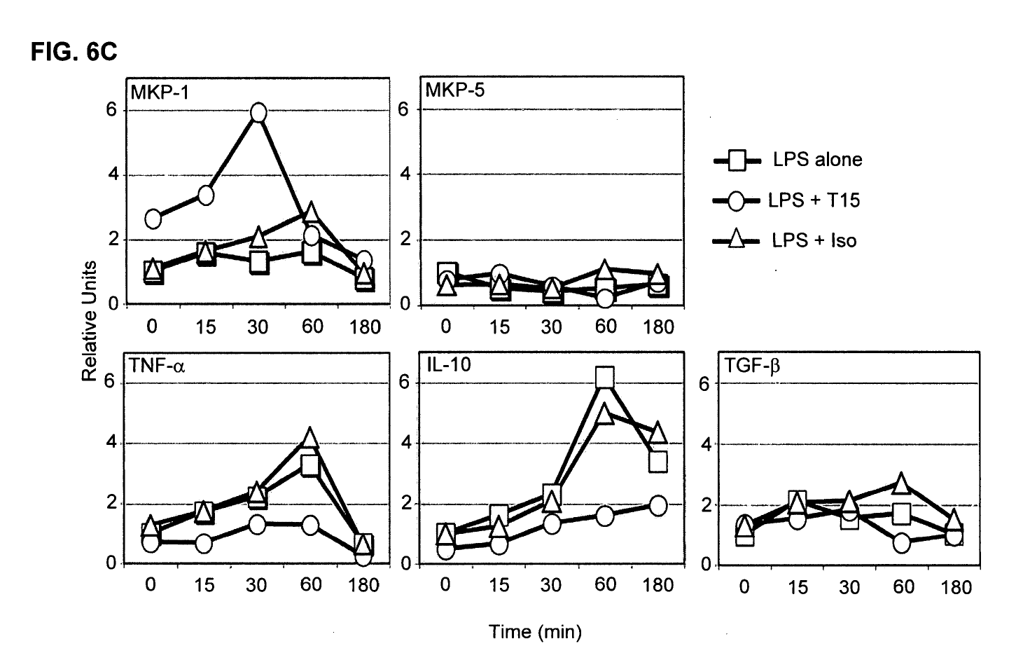

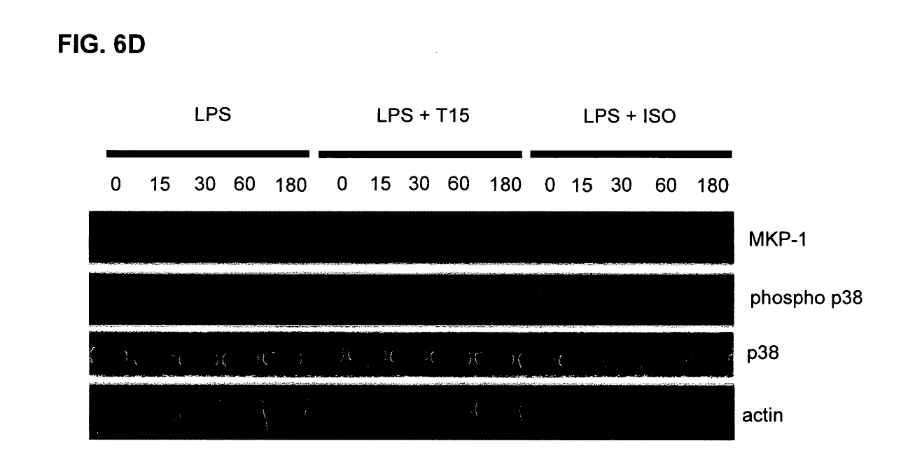

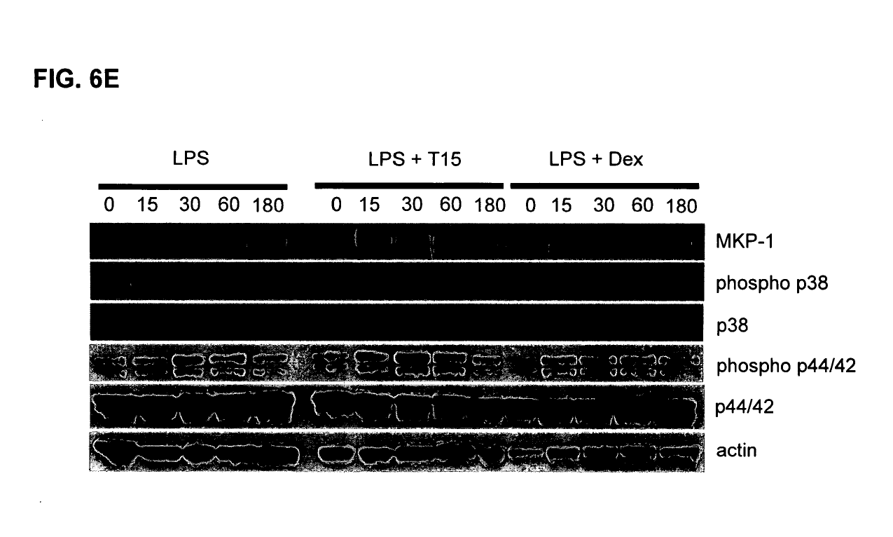

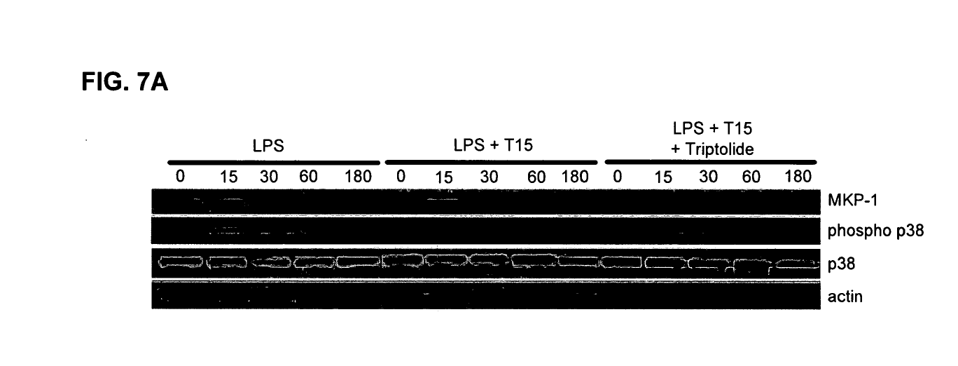

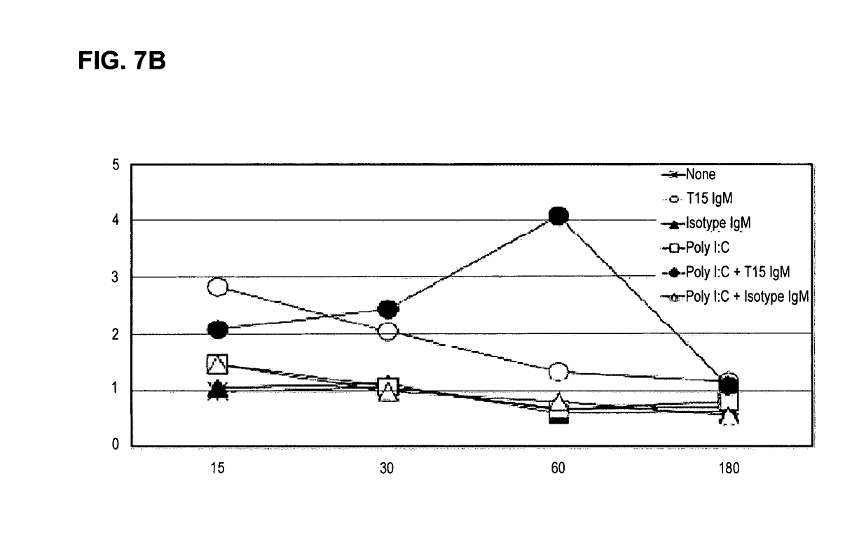

(D) The capacity of antibody with enriched MBL activity to down-modulate the in vitro responses of cultured DC to TLR agonists can be demonstrated (in comparison to the antibody prior to enhancement) by determining the capacity to inhibit lipopolysaccharide (LPS) induced cytokine secretion (see FIGS. 6 and 7). Alternatively, timed cultures are later assessed after mRNA extraction for relative induction of the transcript of the inhibitory MKP-1 gene and the MKP-1 protein product (see FIGS. 7A&7B).

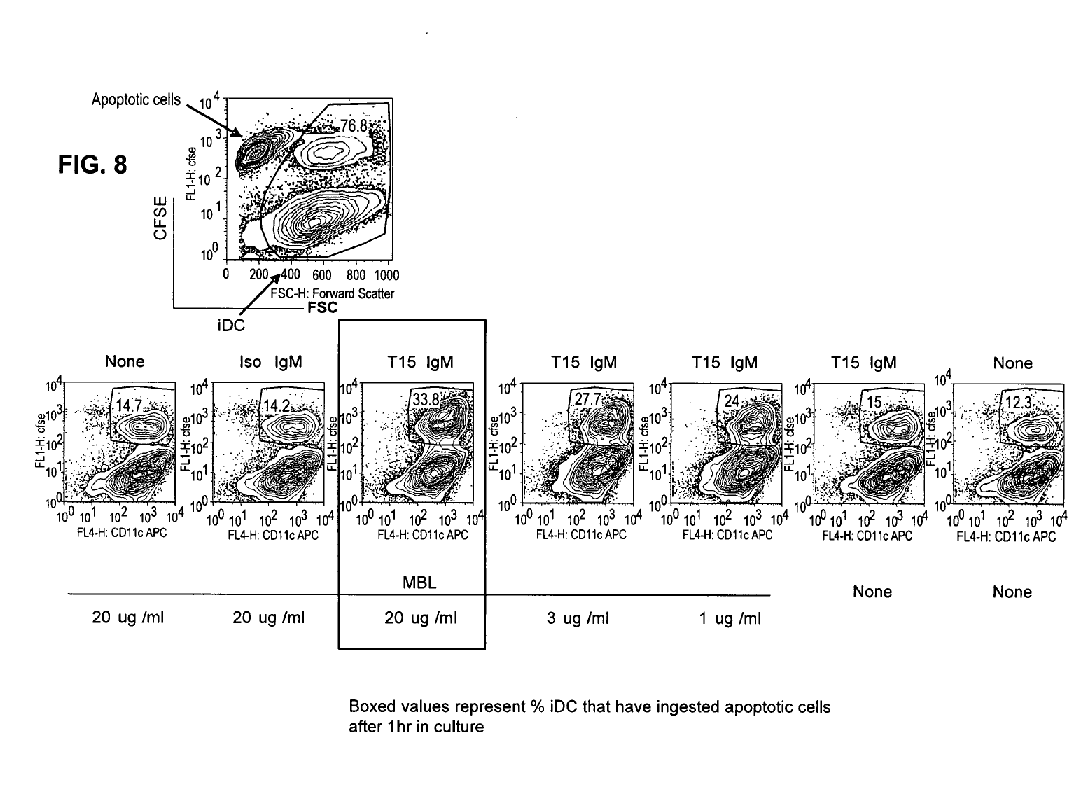

(E) The capacity of antibody with MBL binding activity to enhance phagocytosis of apoptotic cells by immature dendritic cells can be demonstrated, and shown to be MBL dependent (see FIG. 8). These findings are important as apoptotic-cell phagocytosis by immature DC enable constant sampling and presentation of self-antigens in a manner believed essential for the maintenance of immune tolerance⁵⁹. Interactions with apoptotic cells have also been shown to disallow TLR induced activation and maturation, and secretion of pro-inflamamtory cytokines⁴⁷. Hence antibody that binds apoptotic cells, which recruits MBL to the complex, enhances phagocytosis by immature DC of apoptotic cells, which results in immunomodulation and downregulation of DC responses.

It is also possible to directly characterize the N-glycan on the Ig molecule to determine the range of glycan forms that are present. Such services are commercially available from profit and non-profit institutions (e.g., the UCSD Glycotechnology Core Resource).

As IgG constant regions can mediate several different functions, the final effector functions represent the composite of these potential activities. These activities include; the circulating half-life that is mediated by the neonatal FcRn, immune complex mediated activating activities mediated by FcγR, and C1q binding activity, in addition to MBL recruitment. In general, for antibodies that can interact with activating FcγR the absence of the terminal sialic acid alone may enhance FcγR-mediated inflammatory responses⁵⁴. Hence a monoclonal IgG version of T15, or other IgG that binds apoptotic cells, could be made that is devoid of the capacity to activate FcγR but still has the same properties linked to recruitment of MBL as the T15 μM, and this antibody would have anti-inflammatory activity. This could be attained by selecting natural IgG subclass constant regions that do not mediate interactions with FcγR or by manipulating IgG constant region sequences to interfere with the FcγR binding site.

MBL is a soluble factor with the capacity to directly bind onto surfaces and is well known for its contributions to the clearance of microbial pathogens and apoptotic cells. The experimental data provided herein showing that MBL is recruited by T15-Ab to early stage apoptotic cells, and that this is associated with enhanced phagocytosis and reduced inflammation, indicate that MBL deposition in this context serves a primarily pro-phagocytic function. Evidence herein shows that a prototypic antibody, T15, can directly induce deposition of MBL, which can directly enhance cell-corpse clearance by macrophages, as well as enhance the phagocytosis of cell corpses by dendritic cells, and also inhibit the activation and maturation of immature DC and other leukocyte types. T15 can also inhibit the capacity of leukocytes for activation induced cytokine and chemokine production.

The experimental data herein suggests a common pathway for the anti-inflammatory effects of T15-Ab/apoptotic cell complexes and glucocorticoids (FIG. 6E). The current findings therefore show that one mechanism by which T15 affects a cell is the upregulation of the MAP kinase inhibitor, MKP-1, which inhibits leukocyte activation and the production of inflammatory factors. Thus, the antibody compositions of the invention may be used in combination with other agents that work synergistically or additively to increase cellular MKP-1 levels or activity. Such other agents that include, for example, corticosteroids⁷³. The invention may also act through other intracellular pathways.

Also, the compositions of the invention in addition to the antibodies described herein may include other agents that can facilitate the clearance of apoptotic or dying cells. Examples include but are not limited to gas6, protein 2, and their domains that contact Tam receptors Tyro3, Ax1 or Mertk(^{75,76}).

The following examples serve to illustrate the present invention. These examples are in no way intended to limit the scope of the invention.

### Example 1

### A. Methods

Antibodies. T15-IgM (from the EO6 hybridoma)⁶, and the IgM isotype control from the hybridoma, NC17-D8 (gift of L. Arnold, UNC, Chapel Hill N.C.), both express J-chain transcripts. Hybridomas were grown under serum-free conditions in hollow fiber (10,000 MWCO) bioreactors in Hybridoma Serum free media (Invitrogen, Carlsbad Calif.) to a cell density of ˜5−10×10⁸/ml) and then maintained for 30-45 days, by NCCC (Minneapolis, Minn.). Supernatants were purified with a 300 kDa tangential flow filtration (TFF) device, followed by a 10 kDa TFF for further concentration, then dialyzed against PBS pH 7.2, with documented low endotoxin (<0.5 EU/mg), then aliquots stored at −80° C. By native PAGE analysis and western blot, the predominant IgM populations were pentamers with <10% hexamers, without monomeric IgM or low molecular weight species. Antibody assays. Standard sandwich ELISA were performed with precoats of goat anti-IgM, PC-albumin for control antigens, with detection with either biotinylated AB1-2 to detect T15-colonotypic antibodies⁶⁰, or anti-IgM or anti-IgG, as described⁶¹.

Assays were adapted to buffer usage required to detect MBL-binding, as described⁶², with limits of detection of ˜5 ng/ml. In these studies MBL binding by IgG could not be detected in sera either before or after thymocytes immunization. Array studies were performed as described⁶³. Mice, Age and gender-matched adult C57BL/6, congenic B-cell deficient muMT, S107.1 homozygotic knockout mice²², BALB/c and DBA/1 mice were provided by The Jackson Laboratory (Bar Harbor, Me.) or bred under SPF-conditions as supervised by UCSD Animal Care Program. All animal protocols were approved by the UCSD IACUC.

In vitro complement deposition. To induce apoptosis, congenic murine thymocytes either received 600 Rads using a Cs¹³⁷ emission source of radiation, or were treated with 10 uM etoposide, then incubated ON in complete media at 37° C. with 5% CO², then washed three times in media before use. Apoptotic thymocytes were incubated at 37° C. with IgM at 20 ug/ml in Tris-buffered saline with 10 mM CaCl₂, and/or Tris-buffered saline (TBS) with 20% Ig-deficient plasma for complement or TBS alone. After 40 min, cells were washed and studied for apoptosis (7AAD and Annexin V), and with APC-labeled goat anti-IgM, and anti-C1q (goat, Cedarlane Labs), or human recombinant MBL (6 ug/ml) and biotinylated mouse anti-human MBL (clone 131-1), or biotinylated rat antibody to murine MBL A (clone 2B4) or antibody to murine MBL C(clone14D12)⁶², in the presence of Fc block⁴⁰.

Apoptotic clearance assays. Using a standard approach⁶⁴, muMT or RAG2ko mice received thioglycollate treatment and 3d later received i.v. PBS or 1 mg of IgM. After 16 hr, 5×10⁶ SNARF-1 labeled apoptotic or fresh thymocytes were instilled, then peritoneal cells recovered after 10 min. For immunofluorescence microscopic studies, cytospins were prepared and Mφ stained with FITC-anti-F4/80, with >800 Mφ counted per mouse and the proportion determined of recovered Mφ that had ingested (and not just surface bound) one or more labeled thymocytes. While longer time periods were also examined, 10-15 min of in vivo exposure yielded the greatest differences between groups, as previously described (36). While dexamethasone-treated thymocytes yielded similar results, most studies used etoposide for apoptosis induction due to >95% Annexin V+ (i.e., apoptotic) thymocyte yields by flow cytometry. In other studies, to quantitate Mφ uptake, flow cytometric analyses were performed with 7AAD and Annexin V staining of apoptotic thymocytes that were tracked via CD3 (Becton-Dickinson), with peritoneal Mφ detected with FITC-conjugated F4/80 (Caltag).

Bone-marrow derived dendritic cells, Bone marrow cells from C57BL/6 femurs/tibias were washed and cultured in RPMI containing 10% FBS 1% Pen-Strep-Glutamine, GM-CSF (10 ng/ml) and IL-4 (400 pg/ml), replenished on d3⁶⁵. On day 6, DC were selected in the presence of Fc block with magnetic anti-CD11c beads using LS magnetic columns (Milltenyi) to >94% CD11c+ purity. DC were further cultured for 24-48 hrs without/with agonists for TLR3, polyinosinic-cystidic acid (polyl:C)(Amersham) at 3.3 ug/ml; TLR4, LPS (E. coli 055:B5, List Biological Labs) at 0.2 ug/ml; TLR7, imiquimod (Invivogen) at 1 ug/ml; or TLR9, phosphorothioate CpG oligo 1018 at 0.5 ug/ml. Replicate cultures included serial concentrations of T15-IgM or IgM-isotype control. Other cultures included blocking antibody to IL-10 or isotype control (R&D Systems) with Fc block, as per manufacturer's directions. Cultures with T15-Ab blockade with AB1-2 anti-idiotype or isotype control also included Fc block. Some studies instead used STEMSPAN SF Expansion (Stem Cell Technologies) serum-free media. QPCR was performed as previously described⁶¹.

DC phagocytosis of apoptotic cells. To assess the phagocytotic capacity of myeloid DC, DC were prepared from bone marrow of C57BL/6 mice as described above using only GNCSF in serum free media followed by purification with anti-CD11c magnetic beads, Apoptosis was induced in congenic thymocytes by overnight incubation in complete RPMI media with etoposide then washed thrice in PBS before labeling with carboxyfluorescein diacetate succimidylester (CFSE), as per manufacturer's directions (Sigma-Aldrich). DC and CFSE labeled apoptotic cells were placed in serum free media on ice, before mixing in equal numbers, for 0.5×10⁶ of each cell type in final culture volume of 200 ul after addition of IgM and recombinant MBL, as indicated, Replicate cultures were also placed on cytospin preps on coverslips for microscopy studies that confirmed the percentage of DC with ingested apoptotic cells.

DC isolation. Splenic DC were isolated, as reported^{65;65}, then evaluated by flow cytometry for defined DC subsets and maturation/activation markers. Transcript analysis after 6 hr of in vitro stimulation of CD11c-enriched BM-derived DC were performed by Taqman (Applied Biosystems), using manufacturer's directions.

Signal transduction studies, DC lysates were prepared in RIPA buffer or 1% NP40 with protease inhibitors (Santa Cruz Biotech), then run on 4-12% pre-cast gels (Criterion, Biorad) and transferred onto PVDF membrane (Invitrogen). Immunoblots used antibodies to; MKP-1 (sc1102, Santa Cruz), phospho-p38 (9211, Cell Signaling), phospho-ERK (4377, Cell Signaling), p38 (9212, Cell Signaling), ERK (4695, Cell Signaling), phospho-Jnk/SAPK (4688, Cell Signaling), JNK/SAPK (9258, Cell Signaling), detected with anti-rabbit-HRP (Amersham Biosciences).

In vivo challenge assays. Based on pilot studies with outcomes assessed after weekly treatments we selected a 2-wk treatment period, which is also the turnover period of most DC populations from stem cells⁶⁶. Hence, groups of adult C57BL/6 received 3 i.p. infusions (d0, 7 & 14) of 1.5 mg of T15-IgM or isotype control. To assess the role of PC-binding specificity, some groups received 1.5 mg of T15-IgM incubated with 2 mg of PC-BSA for 30 min at RT prior to infusion. On d17, at 18 hr before sacrifice mice received saline or challenge with: polyl:C, 100 ug; LPS, 30 ug; imiquimod, 100 ug or PT CpG oligo 1018, 200 ug, As pilot studies did not demonstrate in vivo activation after imiquimod treatment, 300 ug of SM-360320 was used²⁰, due to 100-fold greater potency. Mice were bled at sacrifice, and suspensions of splenocytes and other lymphoid organs evaluated by flow cytometry using standard antibodies and methods (BD-Pharmingen)^{10;61}.

Antibody immunoassays were performed with PC-BSA, BSA (Sigma) using IgG (sub)class and T15 clonotype-specific antibodies, as previously described⁶¹. Soluble factors in IDC supernatants and sera were evaluated by Luminex assay (Biosource-Invitrogen).

Inflammatory arthritis models. For CIA studies, 8-wk-old DBA/1 mice were immunized with avian CII/CFA (Chondrex) at the tail base on d0 and i.p. boosted on d21 with CII/IFA. Anti-CII antibody levels were assayed, per manufacturer's instructions (Chondrex). For histologic analyses, paws and knees of mice sacrificed on day 44 were decalcified, embedded and sectioned. H&E stained slides were scored for inflammatory infiltrates and joint erosions, and safranin O stained for cartilage damage²¹. Collagen antibody-induced arthritis was induced in BALB/c mice with 2 mg of C11-specific monoclonal IgG cocktail injected i.v. on d0, and 72 h later each animal received 50 ug of LPS E. coli 011B4 i.p. (Arthogen-CIA kit, Chemicon Int.). Different groups received T15-IgM or control IgM at 2 mg, or buffer, given as a pre-treatment and every 7 d thereafter, Clinical arthritis was scored visually from 0 to 4, with a maximum score of 16²¹.

Statistical analysis. Values are reported as mean+/−SEM unless otherwise stated. Significance was assigned for P<0.05 by two-tailed t test, with Welsh correction, or ANOVA, as appropriate (Instat, GraphPad).

### B. Results

### T15-Ab Inhibits In Vivo Inflammatory Responses

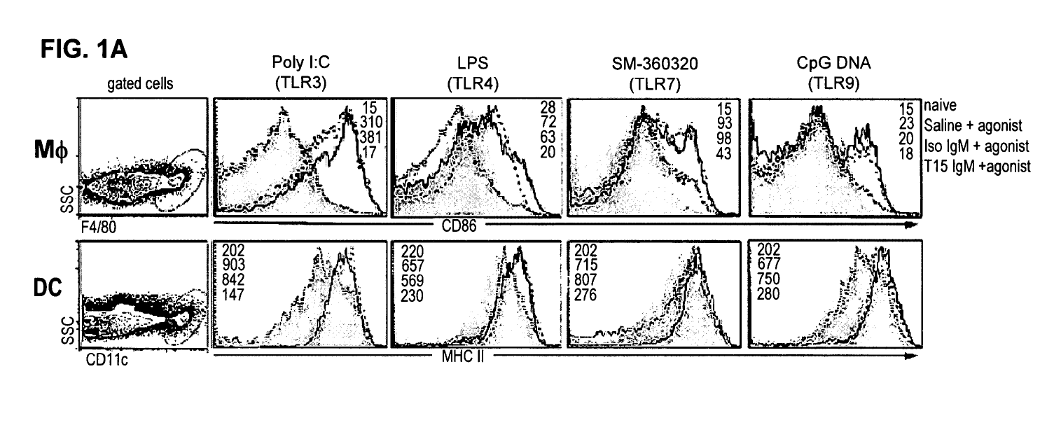

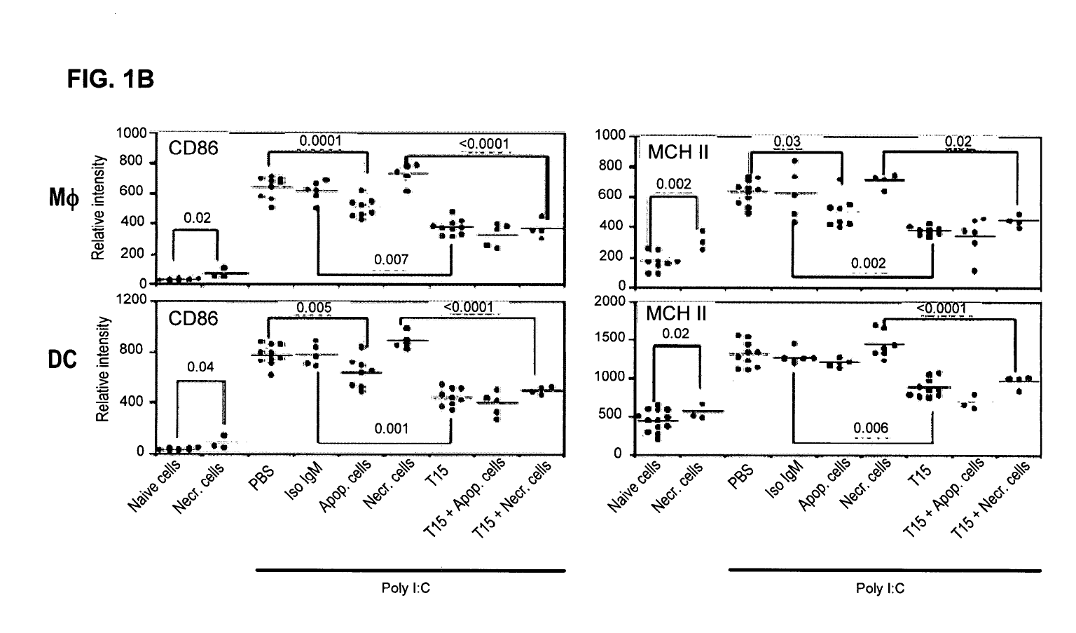

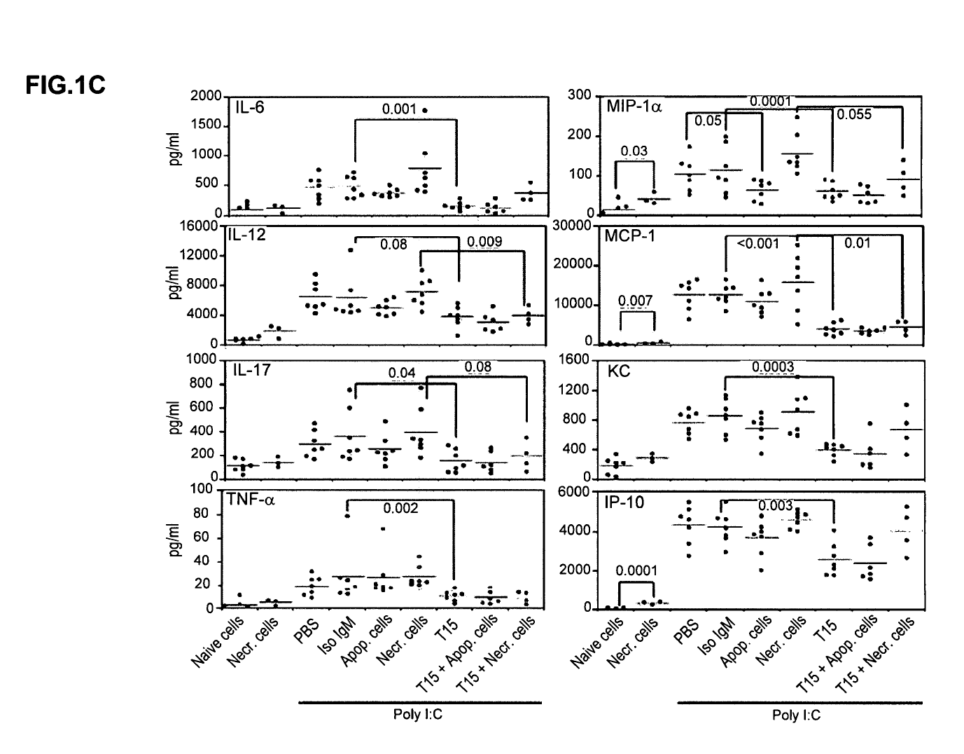

The effects of infusions of purified T15-IgM on in vivo innate immune pro-inflammatory responses were investigated in naïve adult C57BL/6 mice. After two weeks of T15-Ab exposure, which corresponds to the approximate turnover period for DC populations⁶⁶, the T15-Ab group had 17-21% less splenic CD11C^{hi} DCs (P<0.02, N=8 per group) and lower levels of surface-expressed MHC class II. Responses to the TLR agonists, polyl:C (TLR3), LPS (TLR4) and CpG nucleotides (TLR9), were also inhibited by T15-Ab pretreatment, with reduced induction of activation and maturation markers, CD86 and MHC II, on splenic Mφ and CD11c^{hi} DC (FIGS. 1A&1B), Furthermore, T15-Ab also significantly inhibited responses to the potent TLR7 agonist, SM-360320²⁰ (FIG. 1A), as well as poly I:C induction of other co-stimulatory molecules such as CD40, CD80 and B7-DC. T15-Ab treatment also blunted polyl:C-induced blood levels of pro-inflammatory cytokines (IL-6, IL-12, IL-17, TNFα and chemokines (MIP1α, MCP-1, IP-10, KC (FIG. 1C). In addition, T15-Ab treatment significantly reduced the production of IL-6 and IL-12 by peritoneal Mφ. Hence, elevated levels of T15-Ab drastically reduced the in vivo responsiveness of the innate immune system to a range of pro-inflammatory stimuli.

T15 Antibody Protects from Inflammatory Arthritis

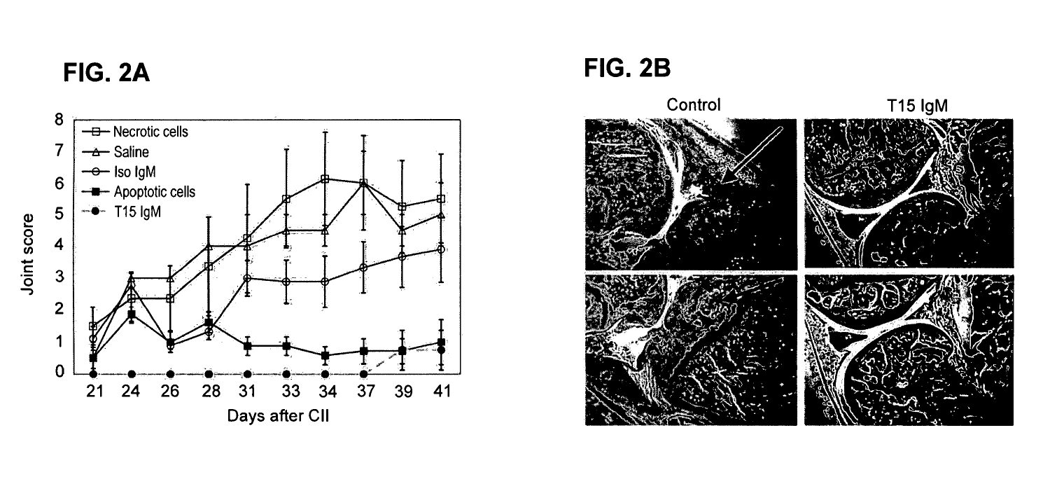

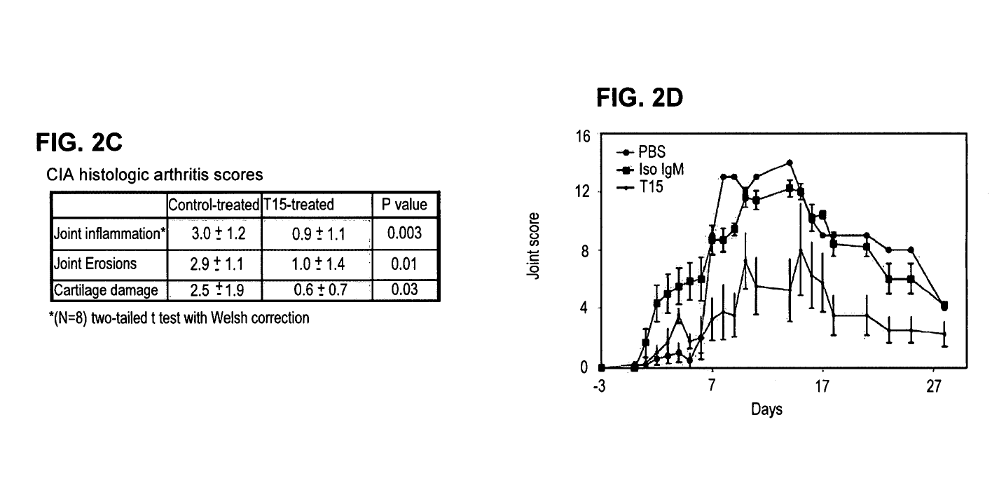

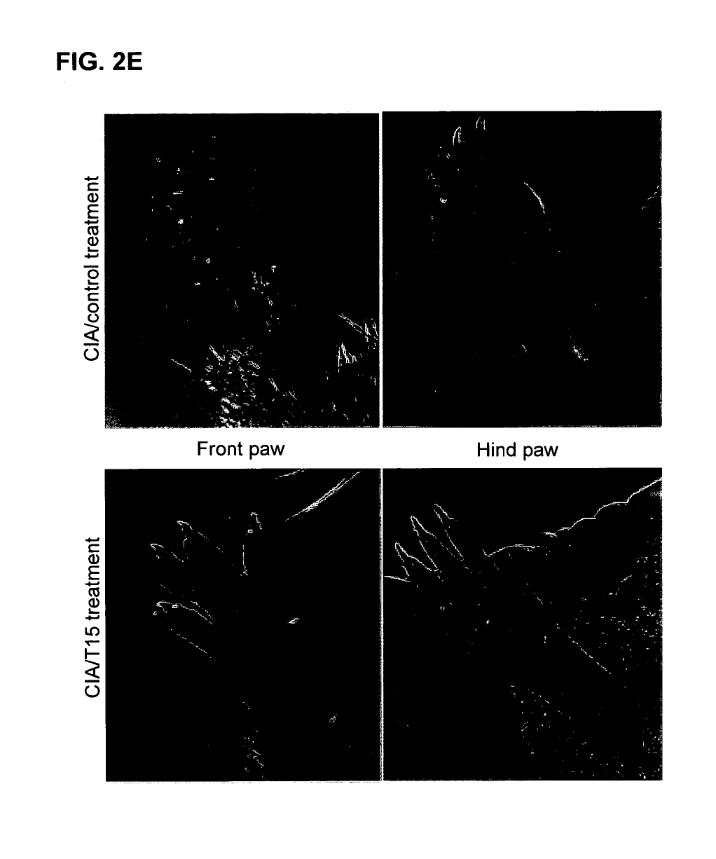

T15-Ab suppression of collagen-induced arthritis (CIA) in DBA/1 mice was studied⁶⁷ (FIG. 2). Pretreatment with the anti-PC antibody significantly reduced clinical disease activity, synovial leukocytic infiltrates, and bone and joint damage (FIG. 2A-2C). There were no differences in total IgG or IgG1 and IgG2a subclass anti-CII levels induced by collagen immunization in the different treatment groups suggesting that T15-Ab was primarily inhibiting the end organ inflammatory response. In other studies, infusions of apoptotic cells into DBA/1 mice yielded increased IgM anti-PC levels and protection from clinical arthritis, while infusions of primary necrotic cells did not (FIG. 2A).

To evaluate for the adaptive immune system's role in this process, the effects of T15-Ab were studied in the model system of passive transfer arthritis induced by anti-CII IgG, in which lymphocytes do not a play central role⁶⁸. Here, T15-Ab treatment significantly diminished joint swelling (FIG. 2D). Together, these findings indicate that the regulatory properties of T15-Ab in these models of arthritis act through the blunting of pro-inflammatory effector mechanisms mediated by the recruitment of IgG-autoantibody immune complexes.

### T15 Antibody Enhances Local Deposition of C1Q and MBL on Apoptotic Cells

To further define the effector functions associated with T15-Ab-mediated interactions, murine MuMT⁶⁹ sera that are deficient in immunoglobulins was used as a source of complement, to study the deposition of C1q onto apoptotic thymocytes (FIG. 3A). Whereas different antigen-specific IgM can vary 20-fold or more in their capacity to recruit C1q⁷⁰, the addition of T15-Ab of the IgM isotype consistently increased the amount of C1q recruitment onto apoptotic cells (FIG. 3A). Notably, T15-Ab was responsible for greater than 3-fold relative increases in C1q deposition on cells at early stages of apoptosis. In contrast, neither T15-Ab nor C1q interacted with freshly isolated healthy thymocytes (FIG. 3A).

Studies were performed to assess the capacity of T15-Ab to recruit MBL. Indeed, solid phase immunoassays showed that both T15-Ab and the IgM-isotype control had dose-dependent binding to recombinant human MBL (FIG. 3B). MBL-binding to T15-Ab was inhibited by mannose or N-acetylglucosamine, but not by N-acetylgalactose, and was also calcium-dependent (FIG. 3B), indicating that the carbohydrate recognition domain of MBL is responsible for these IgM-interactions presumably through Fcμ-associated N-glycans⁵². T15-Ab was capable of concurrent interactions with both PC and MBL (FIG. 3B). This suggests that T15-Ab recruits MBL to apoptotic cells.

Studies were also performed to determine if T15-Ab could promote binding of human recombinant MBL to apoptotic thymocytes. The addition of T15-Ab significantly enhanced MBL deposition with the greatest increases on thymocytes at early stages of apoptosis (FIG. 3C). Further analyses were conducted using murine MBL-specific antibodies⁶² to directly detect mouse MBL deposited on apoptotic cells from MuMT sera. Here, T15-IgM similarly induced the recruitment of both MBL A and C gene products, and these interactions were inhibited by mannose (FIG. 3D). Thus, the T15-Ab recruits both C1q and MBL to primarily early, but also late, apoptotic cells.

Studies of the murine in vivo response to apoptotic cell immunization was used to obtain further insight into the range of antigen-binding specificities on apoptotic cells, and the relationship with MBL binding to T15 Ab therapy. These studies used groups of naïve adult C57BL/6 mice, and also congenic S107.1 homozygotic knockout mice²², which are deficient of the single inherited murine VH gene segment essential for the generation of the VH gene of the T15-Ab. Hence S107.1 ko mice cannot make the T15-Ab or related PC-specific antibodies that use the same VH gene. Mice were given infusions of 2.5×10⁷ thymocytes undergoing etoposide induced apoptosis on day 0, 7 and 14 and subsequently sera samples were taken for analysis. Significantly, ELISA studies showed that apoptotic cells induced high titer responses to both PC-bovine serum albumin (BSA) conjugates and to malondialdehyde-BSA conjugates, by contrast S107.1 ko produced little or no antibodies to PC-BSA or to the PC-determinant containing pneumococcal polysaccharide (C-PS) while antibody responses to MDA-BSA were the same or greater (FIG. 4), Flow cytometric studies confirmed that compared to pre-immunization (naïve) sera from the same mice, apoptotic cells induced greatly increased levels of IgM antibodies that bound to apoptotic thymocytes. To assess binding specificity, sera were first incubated with PC-BSA, MDA-BSA or BSA alone. These studies showed that post-immune IgM anti-apoptotic cell responses in C57BL/6 mice were significantly inhibited by PC-BSA and by MDA-BSA but not by BSA alone, with even greater levels of inhibition by co-incubation by PC-BSA and MDA-BSA (FIGS. 4B&4C). These studies suggest that separate sets of antibodies recognize PC- and MDA-containing determinants on apoptotic cells, and which together represent ˜80% of all IgM anti-apoptotic cell antibodies.

PC-BSA preincubation resulted in little or no inhibition of IgM anti-apoptotic cell responses in S107.1 deficient mice, while MDA-BSA preincubation resulted in the same or greater inhibition of IgM anti-apoptotic cell reactivity in S107.1 deficient mice. Hence the PC-specific response in mice to this apoptotic cell immunization regimen is largely dependent on the production of antibody responses using the S107.1 VH gene segment, while the induction in parallel of IgM response to MDA-containing determinants must use other VH gene segments. Importantly, the level of binding of IgM antibodies to apoptotic responses to PC- and MDA-determinants in both C57BL/6 and S107.1 deficient mice appears directly proportional to the level of MBL binding to apoptotic cells, which is greatly increased compared to the level of binding of MBL from murine Ig-deficient sera to apoptotic cells in the absence of added Ig (FIGS. 4B&4C). Hence IgM antibodies to apoptotic cells recruit MBL to these apoptotic cells, and these include both anti-PC antibodies and those to other apoptotic cell-associated determinant.

The current findings therefore show that the anti-inflammatory activity of antibody to apoptotic cell antigens can be the primary determinant, of the capacity for recruitment of mannose binding lectin and its associated downstream effector functions,

### T15 Antibody Enhances In Vivo Macrophage Clearance of Apoptotic Cells.

Tests were performed to determine whether T15-Ab, because of enhancement of MBL and C1q deposition, could affect in vivo phagocytic clearance. MuMT mice were pretreated with either T15-IgM or control pretreatments, injected i.p. with labeled apoptotic thymocytes, and a short time later examined for the proportion of recovered Mφ with phagocytized thymocytes⁶⁴. T15-IgM treatment resulted in 50-60% increases in the proportion of Mφ that engulfed thymocytes, compared to Isotype control (P<0.0004) or saline treatments (P<0.0001, FIG. 3E-3G). Indeed, T15-Ab coated apoptotic thymocytes formed chains and clusters, which were more rapidly engulfed by peritoneal Mφ (FIG. 3F). Flow cytometric analysis for residual apoptotic cells demonstrated that T15-Ab enhanced the elimination of both early and late stage apoptotic cells (P≦0.004, FIG. 3G). Thus, T15-Ab significantly enhances phagocytosis of apoptotic cells,

### T15 Antibody Inhibits In Vitro Inflammatory Responses of DC

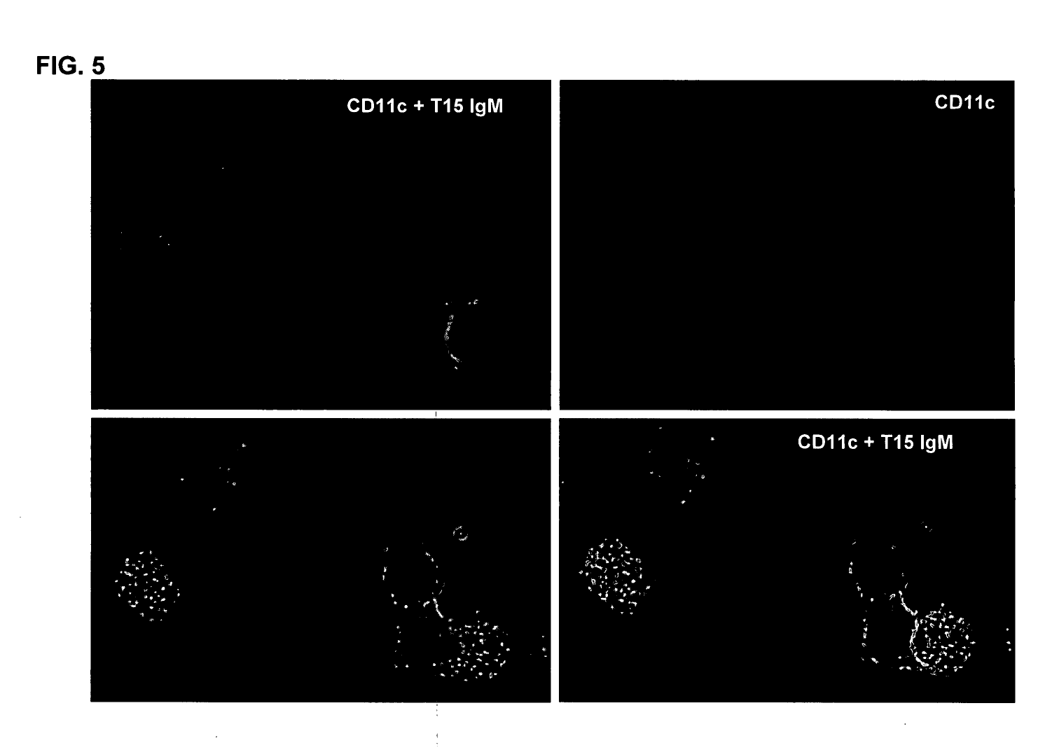

At early stages of differentiation, immature DC share many cell surface receptors as well as the phagocytic capacities of Mφ. Immature DC also produce both C1q⁷¹ and MBL²³ that enhance their capacity to engulf apoptotic cells²³. Using a standard system to generate CD11c-positive immature DC⁶⁵, after 24 hr in culture 10-15% of recovered cellular events were Annexin V-positive apoptotic cells and fragments. When added to these DC cultures, T15-Ab coated these apoptotic DC (and their breakdown products) but not viable DC (FIG. 5). Importantly, T15-Ab-coated apoptotic DC were phagocytosed by viable immature DC, while T15-Ab otherwise had no adverse effects on viability or proliferation. Thus, DC cultures contained substantial amounts of apoptotic cells and debris that form into complexes with the T15 antibody.

Studies were conducted to assess whether T15-Ab by binding to apoptotic material in culture and affecting phagocytosis could modulate in vitro responses of DC to several agonistic TLR ligands, including polyl:C, LPS, imiquimod, and CpG DNA. Indeed, inhibition was documented for surface maturation/activation markers, MHC II, CD40, CD86, and CD80 (FIG. 6A) and for secretion of pro-inflammatory cytokines (TNFα, L-6, IL-12p40/p70), CC chemokines (KC, MCP-1, MIP-1a and CXC chemokine (IP-10) (FIG. 6B). By real-time PCR analysis, T15-Ab also inhibited LPS induction of TNF-α, IL-1b, IL-6, and IL-12 transcripts (FIG. 6C). By contrast, at even high concentrations, a monoclonal IgM isotype control, which showed only minor binding to late-stage apoptotic cells, resulted in limited or no inhibition. Further studies showed that T15-Ab-mediated inhibition of IL-6 production was >80% reduced by a T15-specific anti-idiotypic antibody that blocks the T15 PC-binding site⁶⁰. Hence, the current findings show that the specific interactions of T15-Ab with dead and dying cells can inhibit DC maturation and suppress activation-associated expression of cytokine and chemokine factors. Other types of leukocytes, including but not limited to macrophages, may be similarly affected.

Furthermore, sera obtained after apoptotic-cell treatment had markedly increased levels of IgM-antibodies to apoptotic cells and also suppressed in vitro TLR-mediated activation of cultured DC.

### T15-Ab-Apoptotic Cell Complexes Upregulate MKP-1

To determine the pathways responsible for T15-Ab-mediated inhibitory activities, studies examined expression of IL-10 and TGF-β1, which are both implicated in the inhibitory properties of regulatory DC responses. Neither, however, were induced, at either the transcript or protein level by T15 exposure and, in fact, T15-Ab inhibited the LPS-mediated induction of IL-10 (FIG. 6C). The suppressive effects of T15-Ab were also unimpaired by IL-10 neutralizing antibodies or in DC from IL-10-deficient mice.

Since T15-Ab treatment did not reduce TLR transcript expression in PCs, TLR-related MAP kinase signal transduction pathways were studied to determine if they were affected by T15-Ab. T15-Ab, but not isotype control, inhibited the LPS-induced phosphorylation of p38 MAPK, a key step in TLR-mediated pro-inflammatory responses (FIGS. 6D&6E and FIGS. 7A&7B). Since the dual specificity phosphatase, MKP-1 (also called DUSP-1 and CL100) is a well-recognized master MAPK inhibitor (reviewed in⁷²), its role in this process was examined. Significantly, T15-Ab synergized with LPS or polyl:C for early and high levels of MKP-1 induction, at both the transcript and protein levels (FIG. 6C-6E and FIGS. 7A&7B). This same requirement for co-stimulation with TLR ligands for optimal induction of MKP-1 is also documented for the potent anti-inflammatory glucocorticoid, dexamethasone (FIG. 6)⁷³. Notably, each of these agents alone had little or no early effect on MKP-1 expression. By contrast, levels of the related MKP-5 were unaffected (FIG. 6C). Moreover, confirming the role of MKP-1 in mediating T15-Ab effects, pretreatment with triptolide, which inhibits MKP-1 transcript expression⁷³, blocked the inhibitory effect of T15-Ab on LPS-mediated p38 phosphorylation (FIGS. 7A&7B). Triptolide alone did not affect. DC viability. As for the other primary MAPK pathways, T15-Ab induced modest, but reproducible early and late increases in the phosphorylation of Extracellular signal-regulated kinase (ERK1/2) (FIG. 6E), a known positive regulator of MKP-1 activity⁷², but had no effect on JNK/SAPK expression. Notably, adding an excess of apoptotic cells alone did not inhibit LPS-mediated MAPK activation, as earlier reported⁷⁴, Taken together, the current findings show that interactions with T15-Ab enhance an anti-inflammatory pathway mediated by MKP-1 that is not efficiently induced by apoptotic-cell exposure alone.

As shown in FIG. 8, in vitro studies were also performed that showed that addition of T15-Ab enhanced the capacity of immature dendritic cells to phagocytose labeled apoptotic thymocytes. Significantly, in serum-free media, T15-Ab at 20 ug/ml induced higher rates of phagocytosis than the isotype control. As immature DC are reported to produce MBL²³, there is likely some level of MBL in these cultures even without the addition of MBL. Addition of MBL increased phagocytosis in a dose-dependent manner, with significantly higher levels of phagocytosis with T15-Ab (20 ug/ml) and MBL (1 ug/ml), and the highest level of phagocytosis in cultured immature dendritic cells after the addition of T15-Ab and MBL (20 ug/ml). The relevance of these in vitro culture results is heightened as the levels of T15 IgM and MBL used in these cultures are attainable in vivo. These findings are also important as apoptotic-cell phagocytosis by immature DC enable constant sampling and presentation of self-antigens in a manner believed essential for the maintenance of immune tolerance⁵⁹. Interactions with apoptotic cells have also been shown to disallow TLR induced activation and maturation, and secretion of pro-inflammatory cytokines⁴⁷. Hence antibody that binds apoptotic cells, which recruits MBL to the complex, enhances phagocytosis by immature DC of apoptotic cells, which results in immunomodulation and downregulation of DC responses, including the production of inflammatory cytokines and chemokines

### REFERENCES

1. Marshak-Rothstein, A. Toll-like receptors in systemic autoimmune disease, Nat Rev Immunol, 6(11), 823-35. 2006.
2. Munz, C., Steinman, R. M., and Fujii, S. Dendritic cell maturation by innate lymphocytes: coordinated stimulation of innate and adaptive immunity. J Exp Med. 202(2), 203-7. 2005.
3. Cohn, M., Notani, G., and Rice, S, A. Characterization of the antibody to the C-carbohydrate produced by a transplantable mouse plasmacytoma. Immunochemistry 6(1), 111-23. 1969.
4. Sigal, N. H., Gearhart, P. J., and Klinman, N. R. The frequency of phosphorylcholine-specific B cells in conventional and germfree BALB/C mice. J. Immunol, 114, 1354-1358. 1975.
5. McDaniel, L. S., Benjamin, W. H. J., Forman, C., and Briles, D. E. Blood clearance by anti-phosphocholine antibodies as a mechanism of protection in experimental pneumococcal bacteremia. J. Immunol. 133, 3308-3312. 1984.
6. Shaw, P. X., Horkko, S., Chang, M. K., Curtiss, L. K., Palinski, W., Silverman, G. J., and Witztum, J. L, Natural antibodies with the T15 idiotype may act in atherosclerosis, apoptotic clearance, and protective immunity. J Clin Invest 105(12), 1731-40, 2000.
7, Binder, C. et al. Pneumococcal vaccination decreases atherosclerotic lesion formation: Molecular mimicry between oxidized LDL and Streptococcus pneumoniae. Nature Medicine 9, 736-43 2003.
8, Chang, M. K., Bergmark, C., Laurila, A., Horkko, S., Han, K. H., Friedman, P., Dennis, E. A., and Witztum, J. L. Monoclonal antibodies against oxidized low-density lipoprotein bind to apoptotic cells and inhibit their phagocytosis by elicited macrophages: evidence that oxidation-specific epitopes mediate macrophage recognition, Proc Natl Acad Sci USA 96(11), 6353-8. 1999.
9. Kim, S. J., Gershov, D., Ma, X., Brot, N., and Elkon, K. B. I-PLA(2) activation during apoptosis promotes the exposure of membrane lysophosphatidylcholine leading to binding by natural immunoglobulin M antibodies and complement activation. J Exp Med 196(5), 655-65, 2002.
10. Shaw, P. X., Goodyear, C. S., Chang, M.-K., Witztum, J., and Silverman, G. The Autoreaotivity of Anti-Phosphorylcholine Antibodies for Atherosclerosis-Associated Neo-antigens and Apoptotic Cells, J. Immunol. 170, 6151-7, 2003.
11, Raju, T. S. Terminal sugars of Fc glycans influence antibody effector functions of IgGs. Curr Opin Immunol. 20(4), 471-8, 2008.
12. Sensel, M. G., Kane, L. M., and Morrison, S. L. Amino acid differences in the N-terminus of C(H)2 influence the relative abilities of IgG2 and IgG3 to activate complement. Mol Immunol. 34(14), 1019-29. 1997.
13. Xu, Y., Oomen, R., and Klein, M. H. Residue at position 331 in the IgG1 and IgG4 CH2 domains contributes to their differential ability to bind and activate complement. J Biol. Chem. 269(5), 3469-74. 1994.
14. Arya, S., Chen, F., Spycher, S., Isenman, D. E., Shulman, M. J., and Painter, R, H. Mapping of amino acid residues in the C mu 3 domain of mouse IgM important in macromolecular assembly and complement-dependent cytolysis. J. Immunol. 152(3), 1206-12. 1994.
15. Idusogie, E. E., Wong, P. Y., Presta, L. G., Gazzano-Santoro, H., Totpal, K., Ultsch, M., and Mulkerrin, M. G. Engineered antibodies with increased activity to recruit complement. J Immunol 166(4), 2571-5. 2001.
16. Cocca, B. A., Seal, S, N., D'Agnillo, P., Mueller, Y. M., Katsikis, P. D., Rauch, J., Weigert, M., and Radic, M. Z. Structural basis for autoantibody recognition of phosphatidylserine-beta 2 glycoprotein I and apoptotic cells. Proc Natl Acad Sci USA 98(24), 13826-31, 2001.
17, Shaw, P, X., Horkko, S., Tsimikas, S., Chang, M. K., Palinski, W., Silverman, G, J., Chen, P. P., and Witztum, J. L. Human-derived anti-oxidized LDL autoantibody blocks uptake of oxidized LDL by macrophages and localizes to atherosclerotic lesions in vivo. Arterioscler Thromb Vasc Biol 21(8), 1333-9. 2001.
18, Tyurin, V. A., Tyurina, Y. Y., Kochanek, P. M., Hamilton, R., Dekosky, S. T., Greenberger, J, S., Bayir, H., and Kagan, V. E. Chapter nineteen oxidative lipidomics of programmed cell death. Methods Enzymol. 442:375-93, 2008.
19. Kapralov, A. A., Kurnikov, I. V., Viasova, I, I., Belikova, N. A., Tyurin, V. A., Basova, L. V., Zhao, Q., Tyurina, Y. Y., Jiang, J., Bayir, H., Vladimirov, Y, A., and Kagan, V. E. The hierarchy of structural transitions induced in cytochrome c by anionic phospholipids determines its peroxidase activation and selective peroxidation during apoptosis in cells. Biochemistry, 46(49), 14232-44. 2007.
20. Kurimoto, A., Ogino, T., Ichii, S., Isobe, Y., Tobe, M., Ogita, H., Takaku, H., Sajiki, H., Hirota, K., and Kawakami, H. Synthesis and evaluation of 2-substituted 8-hydroxyadenines as potent interferon, inducers with improved oral bioavaiiabilities. Bioorg Med Chem 12(5), 1091-9. 2004.
21, Simelyte, E., Rosengren, S., Boyle, D. L., Corr, M., Green, D. R., and Firestein, G. S. Regulation of arthritis by p53: critical role of adaptive immunity. Arthritis Rheum, 52, 52(6. 6), 1876-84-1876-84. 2005.
22. Mi, Q. S., Zhou, L., Schulze, D. H., Fischer, R. T., Lustig, A., Rezanka, L. J., Donovan, D. M., Longo, D. L., and Kenny, J. J. Highly reduced protection against Streptococcus pneumoniae after deletion of a single heavy chain gene in mouse. Proc Natl Acad Sci USA 97(11), 6031-6. 2000.
23. Nauta, A. J., Castellano, G., Xu, W., Woltman, A. M., Borrias, M. C., Daha, M. R., van Kooten, C., and Roos, A. Opsonization with C1q and mannose-binding lectin targets apoptotic cells to dendritic cells. J. Immunol. 173(5), 3044-50, 2004.
24. Arnold, J. N., Dwek, R. A., Rudd, P. M., and Sim, R. B. Mannan binding lectin and its interaction with immunoglobulins in health and in disease, Immunol Lett. 106(2), 103-10. 2006.
25. Kabat, E. A., Wu, T. T., Perry, H. M., Gottesman, K. S., and Foeller, C. Sequences of proteins of immunological interest. 91. Bethesda, Md., National Institutes of Health. 1991.
26. Johnson, G. and Wu, T. T. Kabat Database and its applications: future directions. Nucleic Acids Res. 29(1), 205-6, 2001.
27. Paul, W. Fundamental Immunology. Raven Press, NY, N.Y., 1993.
28, Shusta, E. V., Holler, P. D., Kieke, M. C., Kranz, D. M., and Wittrup, K. D. Directed evolution of a stable scaffold for T-cell receptor engineering. Nat. Biotechnol. 18(7), 754-9, 2000.
29, Holler, P. D., Holman, P, O., Shusta, E. V., O'Herrin, S., Wittrup, K. D., and Kranz, D, M. In vitro evolution of a T cell receptor with high affinity for peptide/MHC. Proc Natl Acad Sci USA. 97(10), 5387-92. 2000.
30, Huston, J. S., Levinson, D., Mudgett-Hunter, M., Tai, M. S., Novotny, J., Margolies, M. N., Ridge, R. J., Bruccoleri, R. E., Haber, E., Crea, R., and et al. Protein engineering of antibody binding sites: recovery of specific activity in an anti-digoxin single-chain Fv analogue produced in Escherichia coli. Proc Natl Acad Sci USA. 85(16), 5879-83. 1988.
31, Chothia, C, and Lesk, A. M. Canonical structures for the hypervariable regions of immunoglobulins. J. Mol. Biol. 196, 901-917. 1987.
32. Chothia, C., Lesk, A. M., Tramontano, A., Levitt, M., Smith-Gill, S. J., Air, G., Sheriff, S., Padlan, E, A., Davies, D., and Tulip, W. R, Conformations of immunoglobulin hypervariable regions. Nature 342, 877-883. 1989.
33. Tramontano, A., Chothia, C., and Lesk, A. M. Framework residue 71 is a major determinant of the position and conformation of the second hypervariable region in the VH domains of immunoglobulins. J. Mol. Biol. 215, 175-182. 1990.
34. MacCallum, R. M., Martin, A. C., and Thornton, J. M. Antibody-antigen interactions: contact analysis and binding site topography. J Mol. Biol. 262(5), 732-45. 1996.
35. Riechmann, L., Clark, M., Waldmann, H., and Winter, G. Reshaping human antibodies for therapy. Nature. 332(6162), 323-7, 1988,
36. Rader, C., Cheresh, D. A., and Barbas, C. F. 3rd. A phage display approach for rapid antibody humanization: designed combinatorial V gene libraries. Proc Natl Acad Sci USA. 95(15), 8910-5, 1998.
37. Kim, D. D, and Song, W. C. Membrane complement regulatory proteins, Clin Immunol, 118(2-3), 127-36. 2006.
38, Fraser, I. P., Koziel, H., and Ezekowitz, R. A. The serum mannose-binding protein and the macrophage mannose receptor are pattern recognition molecules that link innate and adaptive immunity. Semin Immunol. 10(5), 303-72. 1998.
39, Takahashi, K. and Ezekowitz, R. A. The role of the mannose-binding lectin in innate immunity. Olin Infect Ds, 41 Suppl 7:S440-4, 2005.
40. Nauta, A. J., Raaschou-Jensen, N., Roos, A., Daha, M. R., Madsen, H. O., Borrias-Essers, M. C., Ryder, L. P., Koch, C., and Garred, P. Mannose-binding lectin engagement with late apoptotic and necrotic cells. Eur J Immunol, 33(10), 2853-63, 2003.
41. Ogden, C. A., deCathelineau, A., Hoffmann, P. R., Bratton, D., Ghebrehiwet, B., Fadok, V, A., and Henson, P, M. C1q and mannose binding lectin engagement of cell surface calreticulin and CD91 initiates macropinocytosis and uptake of apoptotic cells. J Exp Med. 194(6), 781-95. 2001.
42. Stuart, L. M., Takahashi, K., Shi, L., Savill, J., and Ezekowitz, R. A. Mannose-binding lectin-deficient mice display defective apoptotic cell clearance but no autoimmune phenotype. J. Immunol. 174(6), 3220-6. 2005.
43. Fraser, D. A., Bohlson, S, S., Jasinskiene, N., Rawal, N., Palmarini, G., Ruiz, S., Rochford, R., and Tenner, A, J. C1q and MBL, components of the innate immune system, influence monocyte cytokine expression, J Leukoc Biol. 80(1), 107-16, 2006.
44, Bohlson, S. S., Fraser, D. A., and Tenner, A. J. Complement proteins C1q and MBL are pattern recognition molecules that signal immediate and long-term protective immune functions. Mol Immunol. 44(1-3), 33-43. 2007.
45. Gardai, S. J., McPhillips, K. A., Frasch, S. C., Janssen, W. J., Starefeldt, A., Murphy-Ullrich, J, E., Bratton, D. L., Oldenborg, P. A., Michalak, M., and Henson, P. M. Cell-surface calreticulin initiates clearance of viable or apoptotic cells through trans-activation of LRP on the phagocyte, Cell. 123(2), 321-34. 2005.
46, Fadok, V. A., Bratton, D. L., and Henson, P. M. Phagocyte receptors for apoptotic cells: recognition, uptake, and consequences. J Clin Invest 108(7), 957-62. 2001.
47. Sen, P., Wallet, M. A., Yi, Z., Huang, Y., Henderson, M., Mathews, C. E., Earp, H. S., Matsushima, G., Baldwin, A. S, Jr, and Tisch, R. M. Apoptotic cells induce Mer tyrosine kinase-dependent blockade of NF-kappaB activation in dendritic cells. Blood. 109(2), 653-60, 2007.
48, Chapter 7, N-linked glycans. in Essentials of Glycobiology, Eds Varki A. et al. Cold Spring Harbor Laboratories Press, Cold Spring Harbor, N.Y. (1999).
49. Schuster, M., Umana, P., Ferrara, C., Brunker, P., Gerdes, C., Waxenecker, G., Wiederkum, S., Schwager, C., Loibner, H., Himmler, O., and Mudde, G. C. Improved effector functions of a therapeutic monoclonal Lewis Y-specific antibody by glycoform engineering. Cancer Res. 65(17), 7934-41, 2005.
50. Terai, I., Kobayashi, K., Vaerman, J. P., and Mafune, N. Degalactosylated and/or denatured IgA, but not native IgA in any form, bind to mannose-binding lectin, J Immunol, 177(3), 1737-45. 2006.
51. Lund, J., Takahashi, N., Hindley, S., Tyler, R., Goodall, M., and Jefferis, R. Glycosylation of human IgG subclass and mouse IgG2b heavy chains secreted by mouse J558L transfectoma cell lines as chimeric antibodies. Hum Antibodies Hybridomas. 4(1), 20-5. 1993.
52. Arnold, J. N., Wormald, M. R., Suter, D. M., Radcliffe, C. M., Harvey, D. J., Dwek, R. A., Rudd, P. M., and Sim, R. B. Human serum IgM glycosylation: identification of glycoforms that can bind to mannan-binding lectin. J Biol Chem. 280(32), 29080-7, 2005.
53. Malhotra, R., Wormald, M. R., Rudd, P. M., Fischer, P. B., Dwek, R, A., and Sim, R. B. Glycosylation changes of IgG associated with rheumatoid arthritis can activate complement via the mannose-binding protein. Nat. Med. 1(3), 237-43. 1995.
54, Kaneko, Y., Nimmerjahn, F., and Ravetch, J. V. Anti-inflammatory activity of immunoglobulin G resulting from Fc sialylation. Science. 313(5787), 670-3. 2006,
55. Cabrera, G., Cremate, J. A., Valdes, R., Garcia, R., Gonzalez, Y., Montesino, R., Gomez, H., and Gonzalez, M. Influence of culture conditions on the N-glycosylation of a monoclonal antibody specific for recombinant hepatitis B surface antigen. Biotechnol Appl Biochem. 41(Pt 1), 67-76. 2005.
56, Nevens, J, R., Mafia, A. K., Wendt, M, W., and Smith, P. K. Affinity chromatographic purification of immunoglobulin M antibodies utilizing immobilized mannan binding protein. J. Chromatogr. 597(1-2), 247-56. 1992.
57, Paidassi, H., Tacnet-Delorme, P., Garlatti, V., Darnault, C., Ghebrehiwet, B., Gaboriaud, C., Arlaud, G. J., and Frachet, P, C1q binds phosphatidylserine and likely acts as a multiligand-bridging molecule in apoptotic cell recognition. J Immunol, 180(4), 2329-38. 2008.
58. Kuipers, S., Aerts, P, C., Sjoholm, A. G., Harmsen, T, and van Dijk, H. A hemolytic assay for the estimation of functional mannose-binding lectin levels in human serum. J Immunol Methods. 268(2), 149-57. 2002.
59. Steinman, R. M., Hawiger, D., and Nussenzweig, M. C, Tolerogenic dendritic cells. Annu Rev Immunol, 21, 685-711. 2003.
60. Kearney, J. F., Barletta, R., Quan, Z. S., and Quintans, J. Monoclonal vs. heterogeneous anti-H-8 antibodies in the analysis of the anti-phosphorylcholine response in BALB/c mice. Eur. J. Immunol. 11, 877-883. 1981
61, Silverman, G. J., Cary, S, P., Dwyer, D. C., Luo, L., Wagenknecht, R., and Curtiss, V. E. A B cell superantigen-induced persistent “Hole” in the B-1 repertoire, J Exp Med 192(1), 87-98. 2000.
62. Liu, H., Jensen, L., Hansen, S., Petersen, S. V., Takahashi, K., Ezekowitz, A. B., Hansen, F. D., Jensenius, J, C., and Thiel, S. Characterization and quantification of mouse mannan-binding lectins (MBL-A and MBL-C) and study of acute phase responses. Scand J. Immunol. 53(5), 489-97, 2001.
63. Silverman, G. S. R., Germar, K., Goodyear C S, Andrews K A, Ginzler E M, and Tsao B P. Genetic imprinting of autoantibody repertoires in SLE patients. Clin Exp Immunol 153, 102-116 (2008).
64. Taylor, P. R., Carugati, A., Fadok, V. A., Cook, H. T., Andrews, M., Carroll, M. C., Savill, J. S., Henson, P. M., Botto, M., and Walport, M. J. A hierarchical role for classical pathway complement proteins in the clearance of apoptotic cells in vivo. J Exp Med 192(3), 359-66, 2000.
65. Inaba, K., Turley, S., Yamaide, F., Iyoda, T., Mahnke, K., Inaba, M., Pack, M., Subklewe, M., Sauter, B., Sheff, D., Albert, M., Bhardwaj, N., Mellman, I., and Steinman, R. M. Efficient presentation of phagocytosed cellular fragments on the major histocompatibility complex class II products of dendritic cells. J Exp Med 188(11), 2163-73. 1998.
66. Kamath, A. T., Henri, S., Battye, F., Tough, D. F., and Shortman, K. Developmental kinetics and lifespan of dendritic cells in mouse lymphoid organs, Blood. 100(5), 1734-41, 2002.
67, Abdollahi-Roodsaz, S., Joosten, L. A., Roelofs, M. F., Radstake, T. R., Matera, G., Popa, C., van der Meer, J, W., Netea, M. G., and van den Berg, W. B. Inhibition of toll-like receptor 4 breaks the inflammatory loop in autoimmune destructive arthritis. Arthritis Rheum. 56(9), 2957-67. 2007.
68. Nandakumar, K. S., Svensson, L., and Holmdahl, R, Collagen type II-specific monoclonal antibody-induced arthritis in mice: description of the disease and the influence of age, sex, and genes. Am J. Pathol. 163(5), 1827-37. 2003.
69. Kitamura, D., Roes, J., Kuhn, R., and Rajewsky, K. A B cell-deficient mouse by targeted disruption of the membrane exon of the immunoglobulin mu chain gene. Nature 350(6317), 423-6. 1991.
70. Hughey, C. T., Brewer, J. W., Colosia, A. D., Rosse, W. F., and Corley, R. B. Production of IgM hexamers by normal and autoimmune B cells: implications for the physiologic role of hexameric IgM. J Immunol, 161(8), 4091-7. 98.
71. Castellano, G., Woltman, A, M., Nauta, A. J., Roos, A., Trouw, L. A., Seelen, M, A., Schena, F. P., Daha, M. R., and van Kooten, C. Maturation of dendritic cells abrogates C1q production in vivo and in vitro, Blood. 103(10), 3813-20. 2004.
72. Liu, Y., Shepherd, E. G., and Nelin, L. D. MAPK phosphatases-regulating the immune response. Nat Rev Immunol, 7(3), 202-12. 2007.
73. Zhou, Y., Ling, E. A., and Dheen, S. T. Dexamethasone suppresses monocyte chemoattractant protein-1 production via mitogen activated protein kinase phosphatase-1 dependent inhibition of Jun N-terminal kinase and p38 mitogen-activated protein kinase in activated rat microglia. J. Neurochem. 102(3), 667-78. 2007.
74. Sen, P., Wallet, M. A., Yi, Z., Huang, Y., Henderson, M., Mathews, C. E., Earp, H. S., Matsushima, G., Baldwin, A. S, Jr, and Tisch, R. M. Apoptotic cells induce Mer tyrosine kinase-dependent blockade of NF-kappaB activation in dendritic cells, Blood, 109(2), 653-60 2007.
75. Thorp, E. and Tabas, I. Mechanisms and consequences of efferocytosis in advanced atherosclerosis. J Leukoc Biol 86, 2009.
76, Gardai, S. J., et al. Cell-Surface Calreticulin Initiates Clearance of Viable or Apoptotic Cells through trans-Activation of LRP on the Phagocyte. Cell 123:321-34. 2005.

What is claimed is:

1. A non-natural composition comprising monoclonal antibodies, the monoclonal antibodies of the composition having a variable region that binds an antigen exposed on dead or dying cells, and a constant region that comprises sequence from a human heavy chain constant region, said sequence having at least one site which is glycosylated, wherein at least 25% of the monoclonal antibodies of the composition can bind to mannose binding lectin (MBL).

2. The composition of claim 1, wherein the monoclonal antibodies comprise a mixture of different monoclonal antibodies.

3. The composition of claim 1, wherein the monoclonal antibodies comprise a single population of monoclonal antibodies.

4. The composition of claim 1, wherein the monoclonal antibodies are monomeric.

5. The composition of claim 1, wherein the monoclonal antibodies are polymeric.

6. The composition of claim 1, wherein some of the monoclonal antibodies are polymeric and some are monomeric.

7. The composition of claim 1, wherein when said monoclonal antibodies, are monomeric, each monomeric antibody comprises on average at least 1 glycan that has ligands that bind to MBL and wherein when said monoclonal antibodies are polymeric, each polymeric antibody comprises on average two or more glycans that bind MBL.

8. The composition of claim 1, wherein said constant region comprises at least one constant region domain or constant region tailpiece from a human immunoglobulin.

9. The composition of claim 8, Wherein said constant region domain comprises at least one of a CH1, CH2, CH3, or CH4 domain.

10. The composition of claim 1, wherein said constant region comprises sequence from an IgG constant region, IgM constant region or IgA constant region.

11. The composition of claim 1, wherein the constant region is a full length human constant region.

12. The composition of claim 1, wherein said monoclonal antibodies are full length.

13. The composition of claim 1, wherein said monoclonal antibodies are a fragment of a full length antibody.

14. The composition of claim 13, wherein the antibody fragment is a Fab molecule or F(ab′)₂ molecule.

15. The composition of claim 1, wherein said antigen is selected from the group consisting of an antigen having a phosphorylcholine (PC) determinant, an antigen having a phosphatidyl serine (PS) determinant, an antigen having a malondialdehyde (MDA) determinant, and an antigen having a cardiolipin determinant.

16. The composition of claim 1 formulated for administration as a pharmaceutical agent with a suitable pharmaceutically acceptable carrier.

17. The composition of claim 1, wherein said antigen exposed on dead or dying cells is present in atherosclerotic plaque or tissues.

18. The composition of claim 1, wherein the constant regions of the monoclonal antibodies, whether or not the variable region is complexed with said antigen, lacks affinity for one or more Fc receptors.

19. A method for treating a disease in a subject resulting from a pathologic inflammatory condition, comprising administering to the subject an effective amount of the composition of claim 1.

20. The method of claims 19, wherein the pathologic inflammatory condition is an autoimmune disease.

21. The method of claim 19, wherein the pathologic inflammatory condition is atherosclerosis.

22. The method of claim 19 wherein the pathologic inflammatory condition is organ or bone marrow transplantation.

23. The method of claim 19, wherein the pathologic inflammatory condition is cancer.

24. The method of claim 19 further comprising administering to the subject an agent that increases the activity or level of MKP-1 in cells.

25. The method of claim 24, wherein said agent is a corticosteroid.
